(19) United States
(12) Patent Application Publication
(10) Pub. No.: US 2023/0242928 A1
(43) Pub. Date: Aug. 3, 2023

(54) MODULATING NUCLEOTIDE EXPRESSION USING EXPRESSION MODULATING ELEMENTS AND MODIFIED TATA AND USE THEREOF

(71) Applicant: PIONEER HI-BRED INTERNATIONAL, INC., JOHNSTON, IA (US)

(72) Inventors: SHANE E ABBITT, ANKENY, IA (US); MARY J FRANK, DES MOINES, IA (US)

(73) Assignee: PIONEER HI-BRED INTERNATIONAL, INC., JOHNSTON, IA (US)

(21) Appl. No.: 17/905,447

(22) PCT Filed: Mar. 11, 2021
(86) PCT No.: PCT/US2021/021888

§ 371 Date: Sep. 1, 2022

Related U.S. Application Data
(60) Provisional application No. 62/989,011, filed on Mar. 13, 2020.

(51) Int. Cl. C12N 15/82

(52) U.S. Cl. CPC C12N 15/8216; C12N 2320/50

(57) ABSTRACT

The disclosure relates to gene expression modulation elements from plants and TATA box sequences and their use in modulating the expression of one or more heterologous nucleic acid fragments in plants. The disclosure further discloses compositions, polynucleotide constructs, transformed host cells, plants and seeds containing the expression modulating elements and TATA box sequences, and methods for preparing and using the same.

### FIELD

This disclosure relates to a plant regulatory elements and fragments thereof and their use in altering expression of nucleotide sequences in plants.

### REFERENCE TO SEQUENCE LISTING SUBMITTED ELECTRONICALLY

The official copy of the sequence listing is submitted electronically via Patent Center as an ASCII formatted sequence listing with a file named 8206-WO-PCT_Seq List_ST25 created on Oct. 7, 2021 and having a size of 926 bytes. The sequence listing contained in this ASCII formatted document is part of the specification and is herein incorporated by reference in its entirety.

### BACKGROUND

Recent advances in plant genetic engineering have opened new doors to engineer plants to have improved characteristics or traits, such as plant disease resistance, insect resistance, herbicidal resistance, and yield improvement. Appropriate regulatory signals present in proper configurations help obtain the desired expression of a gene of interest. These regulatory signals generally include a promoter region, a 5′ non-translated leader sequence, an intron, and a 3′ transcription termination/polyadenylation sequence.

A combination of TATA box and expression modulating elements that increase or decrease expression of operably linked nucleotide sequences in plants are desired to modulate the expression of one or more genes of interest.

### SUMMARY

The disclosure provides a method of modulating expression of an endogenous polynucleotide in a genomic locus of a plant cell, the method including altering one or more nucleotides in a regulatory region of the genomic locus including the endogenous polynucleotide to create a modified TATA box in the regulatory region, wherein the regulatory region further includes one or more copies of a heterologous expression modulating element (EME). In one embodiment, the TATA box created comprises the sequence “TATA”. In another embodiment, the TATA box created comprises the sequence “TATATATA”.

In one embodiment, the TATA box created is within about 2000 nucleotides from a start codon. In another embodiment, the TATA box created is within about 2000, 1000, 900, 800, 700, 600, 500, 400, 300, 200, 190, 180, 170, 160, 150, 140, 130, 120, 110, 100, 90, 80, 70, 60, 50, 40, 30, 20, 10, or 1 nucleotides from a start codon.

In one embodiment, the TATA box created is within about 500 nucleotides from a transcription start site (TSS). In another embodiment, the TATA box created is within about 500, 400, 300, 200, 190, 180, 170, 160, 150, 140, 130, 120, 110, 100, 90, 80, 70, 60, 50, 40, 30, 20, 10, or 1 nucleotides from a transcription start site (TSS).

In one embodiment, the EME is within about 1 to about 5000 nucleotides from the TATA box sequence. In another embodiment, the EME is within about 250, 240, 230, 220, 210, 200, 190, 180, 170, 160, 150, 140, 130, 120, 110, 100, 90, 80, 70, 60, 50, 40, 30, 20, 10, or 1 nucleotides from the TATA box.

In one embodiment, provided is a method which including altering one or more nucleotides in a regulatory region of the genomic locus including the endogenous polynucleotide to create a modified TATA box in the regulatory region, wherein the altering of one or more nucleotides results in a TATA box comprising the sequence TATA from a different sequence. In another embodiment, the altering of one or more nucleotides results in a TATA box comprising a sequence different from a “TATA” sequence.

Also provided is a method of modulating expression of a polynucleotide encoding a polypeptide in a plant, the method including expressing the polynucleotide by operably linking the polynucleotide with a regulatory element, wherein the regulatory element includes one of more EME and TATA box, wherein the EME is heterologous to the polynucleotide and the EME is heterologous to a promoter functional in the plant.

In one aspect, provide is a method of modulating expression of a polynucleotide encoding a polypeptide in a plant, the method comprising expressing the polynucleotide by operably linking the polynucleotide with a regulatory element, wherein the regulatory element comprises one or more EME and TATA box, wherein the TATA box is heterologous to the polynucleotide and the TATA box is heterologous to a promoter functional in the plant.

In another aspect, provided is a recombinant DNA construct comprising a regulatory element and a polynucleotide sequence, wherein the regulatory element comprises one or more EME and TATA box, wherein the EME is heterologous to the polynucleotide sequence.

In yet another aspect, provided is a plant cell comprising a regulatory element and a polynucleotide sequence, wherein the regulatory element comprises one of more EME and TATA box, wherein the EME is heterologous to the polynucleotide sequence.

Also provided is a method of modulating the expression of a polynucleotide sequence of interest in a plant, the method comprising expressing the polynucleotide sequence, wherein expression of the polynucleotide sequence is regulated by a heterologous regulatory element, wherein the regulatory element comprises one of more EME and TATA box, wherein the EME is heterologous to the polynucleotide sequence.

In one aspect, provided is an isolated polynucleotide comprising a regulatory element and a polynucleotide sequence, wherein the regulatory element comprises one of more EME and TATA box, wherein the EME is heterologous to the polynucleotide sequence, wherein the regulatory element is operably linked to the polynucleotide sequence.

In another aspect, provided is a method of generating a population of activation tagged plants comprising one or more copies of a regulatory element, the method comprising transforming a plurality of plants with a recombinant expression cassette comprising the one or more copies of the regulatory element as an activation tag, wherein the regulatory element comprises one of more EME and TATA box; and generating the population of plants that comprise the activation tag.

In yet another aspect, provided is a method of modulating expression of an endogenous polynucleotide in a plant cell, the method comprising providing a deaminase polypeptide operably associated with a site-specific DNA binding polypeptide, whereby the deaminase polypeptide engineers one or more base changes such that at least one copy of a regulatory element is created in a regulatory region of the endogenous polynucleotide, wherein the regulatory element comprises one of more EME and TATA box, thereby modulating expression of the endogenous polynucleotide in the plant cell.

### DETAILED DESCRIPTION

The disclosure of all patents, patent applications, and publications cited herein are incorporated by reference in their entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

An “isolated polynucleotide” generally refers to a polymer of ribonucleotides (RNA) or deoxyribonucleotides (DNA) that is single- or double-stranded, optionally containing synthetic, non-natural or altered nucleotide bases. An isolated polynucleotide in the form of DNA may be comprised of one or more segments of cDNA, genomic DNA or synthetic DNA.

The terms “polynucleotide”, “polynucleotide sequence”, “nucleic acid sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

A “TATA box” motif refers to a sequence found in many core promoter regions of eukaryotes. The TATA box motif is usually found within 100 nucleotides upstream of the transcription start site. The consensus TATA box sequence comprises the sequence of 5′-TATA-3′. A “modified TATA box” or “optimized TATA box” sequence refers to a TATA box sequence where an endogenous or native sequence that does not contain the “TATA” motif has been altered by one or more nucleotides, for example by substituting an endogenous or native nucleotide or by adding or deleting one or more nucleotides, to produce a the consensus “TATA” sequence or multimer of the consensus “TATA” sequence.

A “de-optimized TATA box” sequence refers to a TATA box sequence where an endogenous or native sequence that contains the “TATA” motif has been altered by one or more nucleotides, for example by substituting an endogenous or native nucleotide or by adding or deleting one or more nucleotides, to produce a sequence other than the consensus “TATA” sequence.

The term “one or more” includes “one or two”, “one, two, or three”, “one, two, three, or four” and “one, two, three, four, or five”, but has generally the meaning of “at least one”. As an example, one or more TATA box motif(s) may be one or two TATA box motif(s), one, two, or three TATA box motif(s), one, two, three, or four TATA box motif(s), one, two, three, four, or five TATA box motif(s), or at least one TATA box motif.

As an example, an optimized plant TATA motif may have a sequence NNTATANN, NNTATATATANN, NNTATATATATATANN, NNTATATANN, or NNTATATATATANN. As an example of TATA optimization, if the native TATA sequence were “TAAA”, and optimized sequence could be “TATA”, or “TATATATA”.

“Expression modulating/modulation element” or “EME” as used herein refers to a nucleotide sequence that up or down-regulates the expression of one or more plant genes. EME may have one or more copies of the same sequence arranged head-to-head, tail-to-head, or head-to-tail or a combination thereof configurations. EMEs are derived from plant sequences, or from bacterial or viral enhancer elements. A list of EMEs are described in Publication No. WO 2018/183878, which is incorporated herein in its entirety.

A regulatory element generally refers to a transcriptional regulatory element involved in regulating the transcription of a nucleic acid molecule such as a gene or a target gene. The regulatory element is a nucleic acid and may include a promoter, an enhancer, an intron, a 5′-untranslated region (5′-UTR, also known as a leader sequence), or a 3′-UTR or a combination thereof. A regulatory element may act in “cis” or “trans”, and generally it acts in “cis”, i.e. it activates expression of genes located on the same nucleic acid molecule, e.g. a chromosome, where the regulatory element is located. The nucleic acid molecule regulated by a regulatory element does not necessarily have to encode a functional peptide or polypeptide, e.g., the regulatory element can modulate the expression of a short interfering RNA or an anti-sense RNA.

An enhancer element is any nucleic acid molecule that increases transcription of a nucleic acid molecule when functionally linked to a promoter regardless of its relative position. An enhancer may be an innate element of the promoter or a heterologous element inserted to enhance the level or tissue-specificity of a promoter.

A repressor (also sometimes called herein silencer) is defined as any nucleic acid molecule which inhibits the transcription when functionally linked to a promoter regardless of relative position.

“Promoter” generally refers to a nucleic acid fragment capable of controlling transcription of another nucleic acid fragment. A promoter generally includes a core promoter (also known as minimal promoter) sequence that includes a minimal regulatory region to initiate transcription, that is a transcription start site. Generally, a core promoter includes a TATA box and a GC rich region associated with a CAAT box or a CCAAT box. These elements act to bind RNA polymerase II to the promoter and assist the polymerase in locating the RNA initiation site. Some promoters may not have a TATA box or CAAT box or a CCAAT box, but instead may contain an initiator element for the transcription initiation site. A core promoter is a minimal sequence required to direct transcription initiation and generally may not include enhancers or other UTRs. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Core promoters are often modified to produce artificial, chimeric, or hybrid promoters, and can further be used in combination with other regulatory elements, such as cis-elements, 5′UTRs, enhancers, or introns, that are either heterologous to an active core promoter or combined with its own partial or complete regulatory elements.

The term “cis-element” generally refers to transcriptional regulatory element that affects or modulates expression of an operably linked transcribable polynucleotide, where the transcribable polynucleotide is present in the same DNA sequence. A cis-element may function to bind transcription factors, which are trans-acting polypeptides that regulate transcription.

“Promoter functional in a plant” is a promoter capable of initiating transcription in plant cells whether or not its origin is from a plant cell.

“Tissue-specific promoter” and “tissue-preferred promoter” are used interchangeably to refer to a promoter that is expressed predominantly but not necessarily exclusively in one tissue or organ, but that may also be expressed in one specific cell.

“Developmentally regulated promoter” generally refers to a promoter whose activity is determined by developmental events.

“Constitutive promoter” generally refers to promoters active in all or most tissues or cell types of a plant at all or most developing stages. As with other promoters classified as “constitutive” (e.g. ubiquitin), some variation in absolute levels of expression can exist among different tissues or stages. The term “constitutive promoter” or “tissue-independent” are used interchangeably herein.

A “heterologous nucleotide sequence” generally refers to a sequence that is not naturally occurring with the EME or the TATA box sequence. While this nucleotide sequence is heterologous to the EME or the TATA box sequence, it may be homologous, or native, or heterologous, or foreign, to the plant host. However, it is recognized that the instant EMEs or TATA box sequences may be used with their native coding sequences to increase or decrease expression resulting in a change in phenotype in the transformed seed. The terms “heterologous nucleotide sequence”, “heterologous sequence”, “heterologous nucleic acid fragment”, and “heterologous nucleic acid sequence” are used interchangeably herein.

A “functional fragment” refers to a portion or subsequence of the sequence described in the present disclosure in which, the ability to modulate gene expression is retained. Fragments can be obtained via methods such as site-directed mutagenesis and synthetic construction. As with the provided promoter sequences described herein, the functional fragments operate to promote the expression of an operably linked heterologous nucleotide sequence, forming a recombinant DNA construct (also, a chimeric gene). For example, the fragment can be used in the design of recombinant DNA constructs to produce the desired phenotype in a transformed plant. Recombinant DNA constructs can be designed for use in co-suppression or antisense by linking a promoter fragment in the appropriate orientation relative to a heterologous nucleotide sequence.

A nucleic acid fragment that is functionally equivalent to the EME and TATA box sequences of the present disclosure is any nucleic acid fragment that is capable of modulating the expression of a coding sequence or functional RNA in a similar manner to the EME and TATA box sequences of the present disclosure.

The polynucleotide sequence of the EME and TATA box sequences of the present disclosure, may be modified or altered to enhance their modulation characteristics. As one of ordinary skill in the art will appreciate, modification or alteration can also be made without substantially affecting the gene expression function. The methods are well known to those of skill in the art. Sequences can be modified, for example by insertion, deletion, or replacement of template sequences through any modification approach.

A “variant promoter” as used herein, is the sequence of the promoter or the sequence of a functional fragment of a promoter containing changes in which one or more nucleotides of the original sequence is deleted, added, and/or substituted, while substantially maintaining promoter function. One or more base pairs can be inserted, deleted, or substituted internally to a promoter. In the case of a promoter fragment, variant promoters can include changes affecting the transcription of a minimal promoter to which it is operably linked. Variant promoters can be produced, for example, by standard DNA mutagenesis techniques or by chemically synthesizing the variant promoter or a portion thereof.

Methods for construction of chimeric and variant EME and TATA box sequences of the present disclosure include, but are not limited to, combining EME elements of different EMEs or duplicating portions or regions of one or more EMEs along with native or modified TATA box sequences. Those of skill in the art are familiar with the standard resource materials that describe specific conditions and procedures for the construction, manipulation, and isolation of macromolecules (e.g., polynucleotide molecules and plasmids), as well as the generation of recombinant organisms and the screening and isolation of polynucleotide molecules. In one embodiment, a TATA box sequence is located about 1, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 300, 350, 400, 500, 600, 700, 800, 900, 1000, or 2000 nucleotides from an ATG start codon. In a further embodiment, an intron sequence may be included between a TATA box sequence and an ATG start codon. In one embodiment, a TATA box sequence is located about 10, 50, 100, 200, 500, 1000, 2000, 3000 nucleotides from an ATG start codon, including an intron sequence. In one aspect of the present disclosure, an EME is located about 1, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 300, 350, 400, 500, 600, 700, 800, 900, 1000, or 2000 nucleotides from an ATG start codon. In a further embodiment, an intron sequence may be included between an EME and an ATG start codon. In one embodiment, an EME is located about 10, 50, 100, 200, 500, 1000, 2000, 3000 nucleotides from an ATG start codon, including an intron sequence. In another aspect of the present disclosure, an EME may be located about 1, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 300, 350, 400, 500, 600, 700, 800, 900, or 1000 nucleotides from a TATA box sequence.

In some aspects of the present disclosure, the promoter fragments can comprise at least about 20 contiguous nucleotides, or at least about 50 contiguous nucleotides, or at least about 75 contiguous nucleotides, or at least about 100 contiguous nucleotides, or at least about 150 contiguous nucleotides, or at least about 200 contiguous nucleotides. In another aspect of the present disclosure, the promoter fragments can comprise at least about 250 contiguous nucleotides, or at least about 300 contiguous nucleotides, or at least about 350 contiguous nucleotides, or at least about 400 contiguous nucleotides, or at least about 450 contiguous nucleotides, or at least about 500 contiguous nucleotides, or at least about 550 contiguous nucleotides, or at least about 600 contiguous nucleotides, or at least about 650 contiguous nucleotides, or at least about 700 contiguous nucleotides, or at least about 750 contiguous nucleotides, or at least about 800 contiguous nucleotides, or at least about 850 contiguous nucleotides, or at least about 900 contiguous nucleotides, or at least about 950 contiguous nucleotides, or at least about 1000 contiguous nucleotides, or at least about 1050 contiguous nucleotides, or at least about 1200, 1300, 1400, 1500, 2000 contiguous nucleotides and further may include an EME and/or a TATA box. The nucleotides of such fragments may comprise the TATA recognition sequence of the particular promoter sequence. The nucleotides of such fragments may further comprise modified TATA recognition sequence. Such fragments may be obtained by use of restriction enzymes to cleave the naturally occurring promoter nucleotide sequences disclosed herein, by synthesizing a nucleotide sequence from the naturally occurring promoter DNA sequence, or may be obtained through the use of PCR technology.

The terms “full complement” and “full-length complement” are used interchangeably herein, and refer to a complement of a given nucleotide sequence, wherein the complement and the nucleotide sequence consist of the same number of nucleotides and are 100% complementary.

The terms “substantially similar” and “corresponding substantially” as used herein refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences.

The transitional phrase “consisting essentially of” generally refers to a composition, method that includes materials, steps, features, components, or elements, in addition to those literally disclosed, provided that these additional materials, steps, features, components, or elements do not materially affect the basic and novel characteristic(s) of the claimed subject matter, e.g., one or more of the claimed expression modulating elements (EMEs) or TATA box sequences.

The isolated promoter sequence comprised in the recombinant DNA construct of the present disclosure can be modified to provide a range of constitutive expression levels of the heterologous nucleotide sequence. Thus, less than the entire promoter regions may be utilized and the ability to drive expression of the coding sequence retained. However, it is recognized that expression levels of the mRNA may be decreased with deletions of portions of the promoter sequences. Likewise, the tissue-independent, constitutive nature of expression may be changed.

Modifications of the isolated promoter sequences of the present disclosure can provide for a range of constitutive expression of the heterologous nucleotide sequence. Thus, they may be modified to be weak constitutive promoters or strong constitutive promoters. Generally, by “weak promoter” is intended a promoter that drives expression of a coding sequence at a low level. By “low level” is intended levels about 1/10,000 transcripts to about 1/100,000 transcripts to about 1/500,000 transcripts. Conversely, a strong promoter drives expression of a coding sequence at high level, or at about 1/10 transcripts to about 1/100 transcripts to about 1/1,000 transcripts. Similarly, a “moderate constitutive” promoter is somewhat weaker than a strong constitutive promoter like the maize ubiquitin promoter.

In addition to modulating gene expression, the expression modulating elements disclosed herein are also useful as probes or primers in nucleic acid hybridization experiments. The nucleic acid probes and primers of the EMEs hybridize under stringent conditions to a target DNA sequence. A “probe” is generally referred to an isolated/synthesized nucleic acid to which, is attached a conventional detectable label or reporter molecule, such as for example, a radioactive isotope, ligand, chemiluminescent agent, bioluminescent molecule, fluorescent label or dye, or enzyme. Such detectable labels may be covalently linked or otherwise physically associated with the probe. “Primers” generally referred to isolated/synthesized nucleic acids that hybridize to a complementary target DNA strand which is then extended along the target DNA strand by a polymerase, e.g., a DNA polymerase. Primer pairs often used for amplification of a target nucleic acid sequence, e.g., by the polymerase chain reaction (PCR) or other conventional nucleic-acid amplification methods. Primers are also used for a variety of sequencing reactions, sequence captures, and other sequence-based amplification methodologies. Primers are generally about 15, 20, 25 nucleotides or more, and probes can also be longer about 30, 40, 50 and up to a few hundred base pairs. Such probes and primers are used in hybridization reactions to target DNA or RNA sequences under high stringency hybridization conditions or under lower stringency conditions, depending on the need.

Moreover, the skilled artisan recognizes that substantially similar nucleic acid sequences encompassed by this disclosure are also defined by their ability to hybridize, under moderately stringent conditions (for example, 0.5×SSC, 0.1% SDS, 60° C.) with the sequences exemplified herein, or to any portion of the nucleotide sequences reported herein and which are functionally equivalent to the promoter of the disclosure. Estimates of such homology are provided by either DNA-DNA or DNA-RNA hybridization under conditions of stringency as is well understood by those skilled in the art (Hames and Higgins, Eds.; In Nucleic Acid Hybridisation; IRL Press: Oxford, U. K., 1985). Stringency conditions can be adjusted to screen for moderately similar fragments, such as homologous sequences from distantly related organisms, to highly similar fragments, such as genes that duplicate functional enzymes from closely related organisms. Post-hybridization washes partially determine stringency conditions. One set of conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. Another set of stringent conditions uses higher temperatures in which the washes are identical to those above except for the temperature of the final two 30 min washes in 0.2×SSC, 0.5% SDS was increased to 60° C. Another set of highly stringent conditions uses two final washes in 0.1×SSC, 0.1% SDS at 65° C.

Preferred substantially similar nucleic acid sequences encompassed by this disclosure are those sequences that are 80% identical to the nucleic acid fragments reported herein or which are 80% identical to any portion of the nucleotide sequences reported herein. More preferred are nucleic acid fragments which are 90% identical to the nucleic acid sequences reported herein, or which are 90% identical to any portion of the nucleotide sequences reported herein. Most preferred are nucleic acid fragments which are 95% identical to the nucleic acid sequences reported herein, or which are 95% identical to any portion of the nucleotide sequences reported herein. It is well understood by one skilled in the art that many levels of sequence identity are useful in identifying related polynucleotide sequences. Useful examples of percent identities are those listed above, or also preferred is any integer percentage from 71% to 100%, such as 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

In one embodiment, the isolated EME sequence comprised in the recombinant DNA construct of the present disclosure comprises a nucleotide sequence having at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95% 96%, 97%, 98%, 99% and 100% sequence identity, based on the Clustal V method of alignment with pairwise alignment default parameters (KTUPLE=2, GAP PENALTY=5, WINDOW=4 and DIAGONALS SAVED=4), when compared to the nucleotide sequences of the present disclosure. It is known to one of skilled in the art that a 5′ UTR region can be altered (deletion or substitutions of bases) or replaced by an alternative 5′UTR while maintaining promoter activity.

A “substantially similar sequence” generally refers to variants of the disclosed sequences such as those that result from site-directed mutagenesis, as well as synthetically derived sequences. A substantially similar sequence of the present disclosure also generally refers to those fragments of a particular promoter nucleotide sequence disclosed herein that operate to promote the constitutive expression of an operably linked heterologous nucleic acid fragment. These promoter fragments comprise at least about 20 contiguous nucleotides, at least about 50 contiguous nucleotides, at least about 75 contiguous nucleotides, preferably at least about 100 contiguous nucleotides of the particular promoter nucleotide sequence disclosed herein or a sequence that is at least 95 to about 99% identical to such contiguous sequences. The nucleotides of such fragments will usually include the TATA recognition sequence (or CAAT box or a CCAAT) of the particular promoter sequence. Such fragments may be obtained by use of restriction enzymes to cleave the naturally occurring promoter nucleotide sequences disclosed herein; by synthesizing a nucleotide sequence from the naturally occurring promoter DNA sequence; or may be obtained through the use of PCR technology. Variants of these promoter fragments, such as those resulting from site-directed mutagenesis, are encompassed by the compositions of the present disclosure.

“Codon degeneracy” generally refers to divergence in the genetic code permitting variation of the nucleotide sequence without affecting the amino acid sequence of an encoded polypeptide. Accordingly, the instant disclosure relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for improved expression in a host cell, it is desirable to design the nucleic acid fragment such that its frequency of codon usage approaches the frequency of preferred codon usage of the host cell.

Sequence alignments and percent identity calculations may be determined using a variety of comparison methods designed to detect similar or identical sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

In one embodiment the % sequence identity is determined over the entire length of the molecule (nucleotide or amino acid). A “substantial portion” of an amino acid or nucleotide sequence comprises enough of the amino acid sequence of a polypeptide or the nucleotide sequence of a gene to afford putative identification of that polypeptide or gene, either by manual evaluation of the sequence by one skilled in the art, or by computer-automated sequence comparison and identification using algorithms such as BLAST (Altschul, S. F. et al., J. Mol. Biol. 215:403-410 (1993)) and Gapped Blast (Altschul, S. F. et al., Nucleic Acids Res. 25:3389-3402 (1997)). BLASTN generally refers to a BLAST program that compares a nucleotide query sequence against a nucleotide sequence database.

“Gene” includes a nucleic acid fragment that expresses a functional molecule such as, but not limited to, a specific protein, including regulatory sequences preceding (5′ non-coding sequences) and following (3′ non-coding sequences) the coding sequence. “Native gene” generally refers to a gene as found in nature with its own regulatory sequences.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas endonuclease system as disclosed herein. A mutated plant is a plant comprising a mutated gene.

“Chimeric gene” or “recombinant expression construct”, which are used interchangeably, includes any gene that is not a native gene, comprising regulatory and coding sequences that are not found together in nature. Accordingly, a chimeric gene may comprise regulatory sequences and coding sequences that are derived from different sources.

“Coding sequence” generally refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

An “intron” is an intervening sequence in a gene that is transcribed into RNA but is then excised in the process of generating the mature mRNA. The term is also used for the excised RNA sequences. An “exon” is a portion of the sequence of a gene that is transcribed and is found in the mature messenger RNA derived from the gene, but is not necessarily a part of the sequence that encodes the final gene product.

The 5′ untranslated region (5′UTR) (also known as a translational leader sequence or leader RNA) is the region of an mRNA that is directly upstream from the initiation codon. This region is involved in the regulation of translation of a transcript by differing mechanisms in viruses, prokaryotes and eukaryotes.

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“RNA transcript” generally refers to a product resulting from RNA polymerase-catalyzed transcription of a DNA sequence. When an RNA transcript is a perfect complimentary copy of a DNA sequence, it is referred to as a primary transcript or it may be an RNA sequence derived from posttranscriptional processing of a primary transcript and is referred to as a mature RNA. “Messenger RNA” (“mRNA”) generally refers to RNA that is without introns and that can be translated into protein by the cell. “cDNA” generally refers to a DNA that is complementary to and synthesized from an mRNA template using the enzyme reverse transcriptase. The cDNA can be single-stranded or converted into the double-stranded by using the Klenow fragment of DNA polymerase I. “Sense” RNA generally refers to RNA transcript that includes mRNA and so can be translated into protein within a cell or in vitro. “Antisense RNA” generally refers to an RNA transcript that is complementary to all or part of a target primary transcript or mRNA and that blocks expression or transcripts accumulation of a target gene. The complementarity of an antisense RNA may be with any part of the specific gene transcript, i.e. at the 5′ non-coding sequence, 3′ non-coding sequence, introns, or the coding sequence. “Functional RNA” generally refers to antisense RNA, ribozyme RNA, or other RNA that may not be translated but yet has an effect on cellular processes.

The term “operably linked” or “functionally linked” generally refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The terms “initiate transcription”, “initiate expression”, “drive transcription”, and “drive expression” are used interchangeably herein and all refer to the primary function of a promoter. As detailed throughout this disclosure, a promoter is a non-coding genomic DNA sequence, usually upstream (5′) to the relevant coding sequence, and its primary function is to act as a binding site for RNA polymerase and initiate transcription by the RNA polymerase. Additionally, there is “expression” of RNA, including functional RNA, or the expression of polypeptide for operably linked encoding nucleotide sequences, as the transcribed RNA ultimately is translated into the corresponding polypeptide.

The term “expression”, as used herein, generally refers to the production of a functional end-product e.g., an mRNA or a protein (precursor or mature).

The term “expression cassette” as used herein, generally refers to a discrete nucleic acid fragment into which a nucleic acid sequence or fragment can be cloned or synthesized through molecular biology techniques.

Expression or overexpression of a gene involves transcription of the gene and translation of the mRNA into a precursor or mature protein. “Antisense inhibition” generally refers to the production of antisense RNA transcripts capable of suppressing the expression of the target protein. “Overexpression” generally refers to the production of a gene product in transgenic organisms that exceeds levels of production in normal or non-transformed organisms. “Co-suppression” generally refers to the production of sense RNA transcripts capable of suppressing the expression or transcript accumulation of identical or substantially similar foreign or endogenous genes (U.S. Pat. No. 5,231,020). The mechanism of co-suppression may be at the DNA level (such as DNA methylation), at the transcriptional level, or at post-transcriptional level.

As stated herein, “suppression” includes a reduction of the level of enzyme activity or protein functionality (e.g., a phenotype associated with a protein) detectable in a transgenic plant when compared to the level of enzyme activity or protein functionality detectable in a non-transgenic or wild type plant with the native enzyme or protein. The level of enzyme activity in a plant with the native enzyme is referred to herein as “wild type” activity. The level of protein functionality in a plant with the native protein is referred to herein as “wild type” functionality. The term “suppression” includes lower, reduce, decline, decrease, inhibit, eliminate and prevent. This reduction may be due to a decrease in translation of the native mRNA into an active enzyme or functional protein. It may also be due to the transcription of the native DNA into decreased amounts of mRNA and/or to rapid degradation of the native mRNA. The term “native enzyme” generally refers to an enzyme that is produced naturally in a non-transgenic or wild type cell. The terms “non-transgenic” and “wild type” are used interchangeably herein.

“Altering expression” or “modulating expression” generally refers to the production of gene product(s) in plants in amounts or proportions that differ significantly from the amount of the gene product(s) produced by the corresponding wild-type plants (i.e., expression is increased or decreased).

“Transformation” as used herein generally refers to both stable transformation and transient transformation.

“Stable transformation” generally refers to the introduction of a nucleic acid fragment into a genome of a host organism resulting in genetically stable inheritance. Once stably transformed, the nucleic acid fragment is stably integrated in the genome of the host organism and any subsequent generation. Host organisms containing the transformed nucleic acid fragments are referred to as “transgenic” organisms. “Transient transformation” generally refers to the introduction of a nucleic acid fragment into the nucleus, or DNA-containing organelle, of a host organism resulting in gene expression without genetically stable inheritance.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

“Genome” as it applies to plant cells encompasses not only chromosomal DNA found within the nucleus, but organelle DNA found within subcellular components (e.g., mitochondrial, plastid) of the cell.

“Genetic modification” generally refers to modification of any nucleic acid sequence or genetic element by insertion, deletion, or substitution of one or more nucleotides in an endogenous nucleotide sequence by genome editing or by insertion of a recombinant nucleic acid, e.g., as part of a vector or construct in any region of the plant genomic DNA by routine transformation techniques. Examples of modification of genetic components include, but are not limited to, promoter regions, 5′ untranslated leaders, introns, genes, 3′ untranslated regions, and other regulatory sequences or sequences that affect transcription or translation of one or more nucleic acid sequences.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

The terms “monocot” and “monocotyledonous plant” are used interchangeably herein. A monocot of the current disclosure includes the Gramineae.

The terms “dicot” and “dicotyledonous plant” are used interchangeably herein. A dicot of the current disclosure includes the following families: Brassicaceae, Leguminosae, and Solanaceae.

“Progeny” comprises any subsequent generation of a plant.

The heterologous polynucleotide can be stably integrated within the genome such that the polynucleotide is passed on to successive generations. The heterologous polynucleotide may be integrated into the genome alone or as part of a recombinant DNA construct. The alterations of the genome (chromosomal or extra-chromosomal) by conventional plant breeding methods, by genome editing procedures that do not result in an insertion of a foreign polynucleotide, or by naturally occurring events such as random cross-fertilization, non-recombinant viral infection, non-recombinant bacterial transformation, non-recombinant transposition, or spontaneous mutation are also methods of modifying a host genome.

“Transient expression” generally refers to the temporary expression of often reporter genes such as β-glucuronidase (GUS), fluorescent protein genes ZS-GREEN1, ZS-YELLOW1 N1, AM-CYAN1, DS-RED in selected certain cell types of the host organism in which the transgenic gene is introduced temporally by a transformation method. The transformed materials of the host organism are subsequently discarded after the transient gene expression assay.

Standard recombinant DNA and molecular cloning techniques used herein are well known in the art and are described more fully in Sambrook, J. et al., In Molecular Cloning: A Laboratory Manual; 2^{nd} ed.; Cold Spring Harbor Laboratory Press: Cold Spring Harbor, N.Y., 1989 (hereinafter “Sambrook et al., 1989”) or Ausubel, F. M., Brent, R., Kingston, R. E., Moore, D. D., Seidman, J. G., Smith, J. A. and Struhl, K., Eds.; In Current Protocols in Molecular Biology; John Wiley and Sons: New York, 1990 (hereinafter “Ausubel et al., 1990”).

“PCR” or “Polymerase Chain Reaction” is a technique for the synthesis of large quantities of specific DNA segments, consisting of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps comprises a cycle.

The terms “plasmid”, “vector” and “cassette” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The term “recombinant DNA construct” or “recombinant expression construct” is used interchangeably and generally refers to a discrete polynucleotide into which a nucleic acid sequence or fragment can be moved. Preferably, it is a plasmid vector or a fragment thereof comprising the promoters of the present disclosure. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain lines displaying the desired expression level and pattern. Such screening may be accomplished by PCR and Southern analysis of DNA, RT-PCR and Northern analysis of mRNA expression, Western analysis of protein expression, or phenotypic analysis.

Various changes in phenotype are of interest including, but not limited to, modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic characteristics and traits such as yield and heterosis increase, the choice of genes for transformation may change accordingly. General categories of genes of interest include, but are not limited to, those genes involved in information, such as zinc fingers, those involved in communication, such as kinases, and those involved in housekeeping, such as heat shock proteins. More specific categories, for example, include, but are not limited to, genes encoding important traits for agronomics, insect resistance, disease resistance, herbicide resistance, sterility, grain or seed characteristics, and commercial products. Genes of interest include, generally, those involved in oil, starch, carbohydrate, or nutrient metabolism as well as those affecting seed size, plant development, plant growth regulation, and yield improvement. Plant development and growth regulation also refer to the development and growth regulation of various parts of a plant, such as the flower, seed, root, leaf and shoot.

Other commercially desirable traits are genes and proteins conferring cold, heat, salt, and drought resistance.

Disease and/or insect resistance genes may encode resistance to pests that have great yield drag such as for example, Northern Corn Leaf Blight, head smut, anthracnose, soybean mosaic virus, soybean cyst nematode, root-knot nematode, brown leaf spot, Downy mildew, purple seed stain, seed decay and seedling diseases caused commonly by the fungi—Pythium sp., Phytophthora sp., Rhizoctonia sp., Diaporthe sp.. Bacterial blight caused by the bacterium Pseudomonas syringae pv. Glycinea. Genes conferring insect resistance include, for example, Bacillus thuringiensis toxic protein genes (U.S. Pat. Nos. 5,366,892; 5,747,450; 5,737,514; 5,723,756; 5,593,881; and Geiser et al (1986) Gene 48:109); lectins (Van Damme et al. (1994) Plant Mol. Biol. 24:825); and the like.

Herbicide resistance traits may include genes coding for resistance to herbicides that act to inhibit the action of acetolactate synthase (ALS), in particular the sulfonylurea-type herbicides (e.g., the acetolactate synthase ALS gene containing mutations leading to such resistance, in particular the S4 and/or HRA mutations). The ALS-gene mutants encode resistance to the herbicide chlorsulfuron. Glyphosate acetyl transferase (GAT) is an N-acetyltransferase from Bacillus lichenformis that was optimized by gene shuffling for acetylation of the broad spectrum herbicide, glyphosate, forming the basis of a novel mechanism of glyphosate tolerance in transgenic plants (Castle et al. (2004) Science 304, 1151-1154).

Genes involved in plant growth and development have been identified in plants. One such gene, which is involved in cytokinin biosynthesis, is isopentenyl transferase (IPT). Cytokinin plays a critical role in plant growth and development by stimulating cell division and cell differentiation (Sun et al. (2003), Plant Physiol. 131: 167-176).

In certain embodiments, the present disclosure contemplates the transformation of a recipient cell with more than one advantageous gene. Two or more genes can be supplied in a single transformation event using either distinct gene-encoding vectors, or a single vector incorporating two or more gene coding sequences. Any two or more genes of any description, such as those conferring herbicide, insect, disease (viral, bacterial, fungal, and nematode), or drought resistance, oil quantity and quality, or those increasing yield or nutritional quality may be employed as desired.

This disclosure concerns a recombinant DNA construct comprising an isolated nucleic acid fragment comprising EME and/or TATA box sequences. This disclosure also concerns a recombinant DNA construct comprising a promoter wherein said promoter consists essentially of a nucleotide sequence comprising EME and/or TATA box sequences, or an isolated polynucleotide comprising a promoter wherein said promoter comprises a nucleotide sequence comprising EME and/or TATA box sequences or a functional fragment thereof.

It is clear from the disclosure set forth herein that one of ordinary skill in the art could perform the following procedure:

1) operably linking the nucleic acid fragments containing the EME and TATA box sequences, intron or the 5′UTR sequences to a suitable reporter gene; there are a variety of reporter genes that are well known to those skilled in the art, including the bacterial GUS gene, the firefly luciferase gene, and the cyan, green, red, and yellow fluorescent protein genes; any gene for which an easy and reliable assay is available can serve as the reporter gene.

2) transforming EME and TATA box sequences, intron or the 5′UTR sequences: reporter gene expression cassettes into an appropriate plant for expression of the promoter. There are a variety of appropriate plants which can be used as a host for transformation that are well known to those skilled in the art, including the dicots, Arabidopsis, tobacco, soybean, oilseed rape, peanut, sunflower, safflower, cotton, tomato, potato, cocoa and the monocots, corn, wheat, rice, barley and palm.

3) testing for expression of the EME and TATA box sequences, intron or the 5′UTR sequences in various cell types of transgenic plant tissues, e.g., leaves, roots, flowers, seeds, transformed with the chimeric EME and TATA box sequences, intron or the 5′UTR sequences: reporter gene expression cassette by assaying for expression of the reporter gene product.

In another aspect, this disclosure concerns a recombinant DNA construct comprising at least one heterologous nucleic acid fragment operably linked to any promoter, or combination of promoter elements, of the present disclosure. Recombinant DNA constructs can be constructed by operably linking the nucleic acid fragment of the disclosure EME and TATA box sequences or a functional fragment thereof. Any heterologous nucleic acid fragment can be used to practice the disclosure. The selection will depend upon the desired application or phenotype to be achieved. The various nucleic acid sequences can be manipulated so as to provide for the nucleic acid sequences in the proper orientation. It is believed that various combinations of promoter elements as described herein may be useful in practicing the present disclosure.

In another aspect, this disclosure concerns a recombinant DNA construct comprising at least one gene that provides drought tolerance operably linked to EME and TATA box sequences or a fragment, or combination of promoter elements, of the present disclosure. In another aspect, this disclosure concerns a recombinant DNA construct comprising at least one gene that provides insect resistance operably linked to EME and TATA box sequences or a fragment, or combination of promoter elements, of the present disclosure. In another aspect, this disclosure concerns a recombinant DNA construct comprising at least one gene that increases nitrogen use efficiency and/or yield, operably linked to EME and TATA box sequences or a fragment, or combination of promoter elements, of the present disclosure. In another aspect, this disclosure concerns a recombinant DNA construct comprising at least one gene that provides herbicide resistance operably linked to EME and TATA box sequences or a fragment, or combination of promoter elements, of the present disclosure.

In another embodiment, this disclosure concerns host cells comprising either the recombinant DNA constructs of the disclosure as described herein or isolated polynucleotides of the disclosure as described herein. Examples of host cells which can be used to practice the disclosure include, but are not limited to, yeast, bacteria, and plants.

Plasmid vectors comprising the instant recombinant DNA construct can be constructed. The choice of plasmid vector is dependent upon the method that will be used to transform host cells. The skilled artisan is well aware of the genetic elements that must be present on the plasmid vector in order to successfully transform, select and propagate host cells containing the chimeric gene.

### I. Gene Editing

In some embodiments, gene editing may be facilitated through the induction of a double-stranded break (DSB) in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), and the like. In some embodiments, the introduction of a DSB can be combined with the introduction of a polynucleotide modification template.

A polynucleotide modification template can be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

The polynucleotide modification template can be introduced into a cell as a single stranded polynucleotide molecule, a double stranded polynucleotide molecule, or as part of a circular DNA (vector DNA). The polynucleotide modification template can also be tethered to the guide RNA and/or the Cas endonuclease. Tethered DNAs can allow for co-localizing target and template DNA, useful in genome editing and targeted genome regulation, and can also be useful in targeting post-mitotic cells where function of endogenous HR machinery is expected to be highly diminished (Mali et al. 2013 Nature Methods Vol. 10: 957-963.) The polynucleotide modification template may be present transiently in the cell or it can be introduced via a viral replicon.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

TAL effector nucleases (TALEN) are a class of sequence-specific nucleases that can be used to make double-strand breaks at specific target sequences in the genome of a plant or other organism. (Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H—N—H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognized a sequence of 9 contiguous nucleotides, with a dimerization requirement of the nuclease, two sets of zinc finger triplets are used to bind an 18 nucleotide recognition sequence.

Genome editing using DSB-inducing agents, such as Cas9-gRNA complexes, has been described, for example in U.S. Patent Application US 2015-0082478 A1, published on Mar. 19, 2015, WO2015/026886 A1, published on Feb. 26, 2015, WO2016007347, published on Jan. 14, 2016, and WO201625131, published on Feb. 18, 2016, all of which are incorporated by reference herein.

The term “Cas gene” herein refers to a gene that is generally coupled, associated or close to, or in the vicinity of flanking CRISPR loci in bacterial systems. The terms “Cas gene”, “CRISPR-associated (Cas) gene” are used interchangeably herein. The term “Cas endonuclease” herein refers to a protein encoded by a Cas gene. A Cas endonuclease herein, when in complex with a suitable polynucleotide component, is capable of recognizing, binding to, and optionally nicking or cleaving all or part of a specific DNA target sequence. A Cas endonuclease described herein comprises one or more nuclease domains. Cas endonucleases of the disclosure includes those having a HNH or HNH-like nuclease domain and/or a RuvC or RuvC-like nuclease domain. A Cas endonuclease of the disclosure includes a Cas9 protein, a Cpf1 protein, a C2c1 protein, a C2c2 protein, a C2c3 protein, Cas3, Cas 5, Cas7, Cas8, Cas10, or complexes of these.

In addition to the double-strand break inducing agents, site-specific base conversions can also be achieved to engineer one or more nucleotide changes to create one or more EMEs described herein into the genome. These include for example, a site-specific base edit mediated by an C·G to T·A or an A·T to G·C base editing deaminase enzymes (Gaudelli et al., Programmable base editing of A·T to G·C in genomic DNA without DNA cleavage.” Nature (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533 (7603) (2016):420-4. Catalytically dead dCas9 fused to a cytidine deaminase or an adenine deaminase protein becomes a specific base editor that can alter DNA bases without inducing a DNA break. Base editors convert C->T (or G->A on the opposite strand) or an adenine base editor that would convert adenine to inosine, resulting in an A->G change within an editing window specified by the gRNA.

As used herein, the terms “guide polynucleotide/Cas endonuclease complex”, “guide polynucleotide/Cas endonuclease system”, “guide polynucleotide/Cas complex”, “guide polynucleotide/Cas system”, “guided Cas system” are used interchangeably herein and refer to at least one guide polynucleotide and at least one Cas endonuclease that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double strand break) the DNA target site. A guide polynucleotide/Cas endonuclease complex herein can comprise Cas protein(s) and suitable polynucleotide component(s) of any of the four known CRISPR systems (Horvath and Barrangou, 2010, Science 327:167-170) such as a type I, II, or III CRISPR system. A Cas endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas protein. Such recognition and cutting of a target sequence by a Cas endonuclease typically occurs if the correct protospacer-adjacent motif (PAM) is located at or adjacent to the 3′ end of the DNA target sequence. Alternatively, a Cas protein herein may lack DNA cleavage or nicking activity, but can still specifically bind to a DNA target sequence when complexed with a suitable RNA component. (See also U.S. Patent Application US 2015-0082478 A1, published on Mar. 19, 2015 and US 2015-0059010 A1, published on Feb. 26, 2015, both are hereby incorporated in its entirety by reference).

A guide polynucleotide/Cas endonuclease complex can cleave one or both strands of a DNA target sequence. A guide polynucleotide/Cas endonuclease complex that can cleave both strands of a DNA target sequence typically comprise a Cas protein that has all of its endonuclease domains in a functional state (e.g., wild type endonuclease domains or variants thereof retaining some or all activity in each endonuclease domain). Non-limiting examples of Cas9 nickases suitable for use herein are disclosed in U.S. Patent Appl. Publ. No. 2014/0189896, which is incorporated herein by reference.

Other Cas endonuclease systems have been described in PCT patent applications PCT/US16/32073, filed May 12, 2016 and PCT/US16/32028 filed May 12, 2016, both applications incorporated herein by reference.

“Cas9” (formerly referred to as Cas5, Csn1, or Csx12) herein refers to a Cas endonuclease of a type II CRISPR system that forms a complex with a crNucleotide and a tracrNucleotide, or with a single guide polynucleotide, for specifically recognizing and cleaving all or part of a DNA target sequence. Cas9 protein comprises a RuvC nuclease domain and an HNH (H—N—H) nuclease domain, each of which can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al, Cell 157:1262-1278). A type II CRISPR system includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA.

Any guided endonuclease can be used in the methods disclosed herein. Such endonucleases include, but are not limited to Cas9 and Cpf1 endonucleases. Many endonucleases have been described to date that can recognize specific PAM sequences (see for example—Jinek et al. (2012) Science 337 p 816-821, PCT patent applications PCT/US16/32073, filed May 12, 2016 and PCT/US16/32028 filed May 12, 2016 and Zetsche B et al. 2015. Cell 163, 1013) and cleave the target DNA at a specific positions. It is understood that based on the methods and embodiments described herein utilizing a guided Cas system one can now tailor these methods such that they can utilize any guided endonuclease system.

As used herein, the term “guide polynucleotide”, relates to a polynucleotide sequence that can form a complex with a Cas endonuclease and enables the Cas endonuclease to recognize, bind to, and optionally cleave a DNA target site. The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA” or “gRNA” (See also U.S. Patent Application US 2015-0082478 A1, published on Mar. 19, 2015 and US 2015-0059010 A1, published on Feb. 26, 2015, both are hereby incorporated in its entirety by reference).

The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. The single guide polynucleotide being comprised of sequences from the crNucleotide and the tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double strand break) the target site. (See also U.S. Patent Application US 2015-0082478 A1, published on Mar. 19, 2015 and US 2015-0059010 A1, published on Feb. 26, 2015, both are hereby incorporated in its entirety by reference.)

The term “variable targeting domain” or “VT domain” is used interchangeably herein and includes a nucleotide sequence that can hybridize (is complementary) to one strand (nucleotide sequence) of a double strand DNA target site. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” (of a guide polynucleotide) is used interchangeably herein and includes a nucleotide sequence that interacts with a Cas endonuclease polypeptide. A CER domain comprises a tracrNucleotide mate sequence followed by a tracrNucleotide sequence. The CER domain can be composed of a DNA sequence, a RNA sequence, a modified DNA sequence, a modified RNA sequence (see for example US 2015-0059010 A1, published on Feb. 26, 2015, incorporated in its entirety by reference herein), or any combination thereof.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another embodiment, the nucleotide sequence linking the crNucleotide and the tracrNucleotide of a single guide polynucleotide can comprise a tetraloop sequence, such as, but not limiting to a GAAA tetraloop sequence.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site.

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site. A guide RNA/Cas endonuclease complex herein can comprise Cas protein(s) and suitable RNA component(s) of any of the four known CRISPR systems (Horvath and Barrangou, 2010, Science 327:167-170) such as a type I, II, or III CRISPR system. A guide RNA/Cas endonuclease complex can comprise a Type II Cas9 endonuclease and at least one RNA component (e.g., a crRNA and tracrRNA, or a gRNA). (See also U.S. Patent Application US 2015-0082478 A1, published on Mar. 19, 2015 and US 2015-0059010 A1, published on Feb. 26, 2015, both are hereby incorporated in its entirety by reference).

The guide polynucleotide can be introduced into a cell transiently, as single stranded polynucleotide or a double stranded polynucleotide, using any method known in the art such as, but not limited to, particle bombardment, Agrobacterium transformation or topical applications. The guide polynucleotide can also be introduced indirectly into a cell by introducing a recombinant DNA molecule (via methods such as, but not limited to, particle bombardment or Agrobacterium transformation) comprising a heterologous nucleic acid fragment encoding a guide polynucleotide, operably linked to a specific promoter that is capable of transcribing the guide RNA in said cell. The specific promoter can be, but is not limited to, a RNA polymerase III promoter, which allow for transcription of RNA with precisely defined, unmodified, 5′- and 3′-ends (DiCarlo et al., Nucleic Acids Res. 41: 4336-4343; Ma et al., Mol. Ther. Nucleic Acids 3: e161) as described in WO2016025131, published on Feb. 18, 2016, incorporated herein in its entirety by reference.

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, or any other DNA molecule in the genome (including chromosomal, choloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. Cells include, but are not limited to, human, non-human, animal, bacterial, fungal, insect, yeast, non-conventional yeast, and plant cells as well as plants and seeds produced by the methods described herein. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

Methods for “modifying a target site” and “altering a target site” are used interchangeably herein and refer to methods for producing an altered target site.

The length of the target DNA sequence (target site) can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other Cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by an Cas endonuclease. Assays to measure the single or double-strand break of a target site by an endonuclease are known in the art and generally measure the overall activity and specificity of the agent on DNA substrates containing recognition sites.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

The terms “targeting”, “gene targeting” and “DNA targeting” are used interchangeably herein. DNA targeting herein may be the specific introduction of a knock-out, edit, or knock-in at a particular DNA sequence, such as in a chromosome or plasmid of a cell. In general, DNA targeting can be performed herein by cleaving one or both strands at a specific DNA sequence in a cell with an endonuclease associated with a suitable polynucleotide component. Such DNA cleavage, if a double-strand break (DSB), can prompt NHEJ or HDR processes which can lead to modifications at the target site.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide an guidepolynucleotide/Cas endonuclease complex to a unique DNA target site.

The terms “knock-out”, “gene knock-out” and “genetic knock-out” are used interchangeably herein. A knock-out represents a DNA sequence of a cell that has been rendered partially or completely inoperative by targeting with a Cas protein; such a DNA sequence prior to knock-out could have encoded an amino acid sequence, or could have had a regulatory function (e.g., promoter), for example. A knock-out may be produced by an indel (insertion or deletion of nucleotide bases in a target DNA sequence through NHEJ), or by specific removal of sequence that reduces or completely destroys the function of sequence at or near the targeting site.

The guide polynucleotide/Cas endonuclease system can be used in combination with a co-delivered polynucleotide modification template to allow for editing (modification) of a genomic nucleotide sequence of interest. (See also U.S. Patent Application US 2015-0082478 A1, published on Mar. 19, 2015 and WO2015/026886 A1, published on Feb. 26, 2015, both are hereby incorporated in its entirety by reference.)

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (by HR, wherein a suitable donor DNA polynucleotide is also used). Examples of knock-ins are a specific insertion of a heterologous amino acid coding sequence in a coding region of a gene, or a specific insertion of a transcriptional regulatory element in a genetic locus.

Various methods and compositions can be employed to obtain a cell or organism having a polynucleotide of interest inserted in a target site for a Cas endonuclease. Such methods can employ homologous recombination to provide integration of the polynucleotide of Interest at the target site. In one method provided, a polynucleotide of interest is provided to the organism cell in a donor DNA construct. As used herein, “donor DNA” is a DNA construct that comprises a polynucleotide of Interest to be inserted into the target site of a Cas endonuclease. The donor DNA construct further comprises a first and a second region of homology that flank the polynucleotide of Interest. The first and second regions of homology of the donor DNA share homology to a first and a second genomic region, respectively, present in or flanking the target site of the cell or organism genome. By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

The amount of sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some embodiments the regions of homology share significant sequence homology to the genomic sequence immediately flanking the target site, it is recognized that the regions of homology can be designed to have sufficient homology to regions that may be further 5′ or 3′ to the target site. In still other embodiments, the regions of homology can also have homology with a fragment of the target site along with downstream genomic regions. In one embodiment, the first region of homology further comprises a first fragment of the target site and the second region of homology comprises a second fragment of the target site, wherein the first and second fragments are dissimilar.

As used herein, “homologous recombination” includes the exchange of DNA fragments between two DNA molecules at the sites of homology.

Further uses for guide RNA/Cas endonuclease systems have been described (See U.S. Patent Application US 2015-0082478 A1, published on Mar. 19, 2015, WO2015/026886 A1, published on Feb. 26, 2015, US 2015-0059010 A1, published on Feb. 26, 2015, U.S. application 62/023,246, filed on Jul. 7, 2014, and U.S. application 62/036,652, filed on Aug. 13, 2014, all of which are incorporated by reference herein) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

In an embodiment, through genome editing approaches described herein and those available to one of ordinary skill in the art, specific motifs of one or more regulatory elements of the EMEs disclosed herein can be engineered to modulate the expression of one or more host plant endogenous genes.

Methods for transforming dicots, primarily by use of Agrobacterium tumefaciens, and obtaining transgenic plants have been published, among others, for cotton (U.S. Pat. Nos. 5,004,863, 5,159,135); soybean (U.S. Pat. Nos. 5,569,834, 5,416,011); Brassica (U.S. Pat. No. 5,463,174); peanut (Cheng et al., Plant Cell Rep. 15:653-657 (1996), McKently et al., Plant Cell Rep. 14:699-703 (1995)); papaya (Ling et al., Bio/technology 9:752-758 (1991)); and pea (Grant et al., Plant Cell Rep. 15:254-258 (1995)). For a review of other commonly used methods of plant transformation see Newell, C. A., Mol. Biotechnol. 16:53-65 (2000). One of these methods of transformation uses Agrobacterium rhizogenes (Tepfler, M. and Casse-Delbart, F., Microbiol. Sci. 4:24-28 (1987)). Transformation of soybeans using direct delivery of DNA has been published using PEG fusion (PCT Publication No. WO 92/17598), electroporation (Chowrira et al., Mol. Biotechnol. 3:17-23 (1995); Christou et al., Proc. Natl. Acad. Sci. U S.A. 84:3962-3966 (1987)), microinjection, or particle bombardment (McCabe et al., Biotechnology 6:923-926 (1988); Christou et al., Plant Physiol. 87:671-674 (1988)).

There are a variety of methods for the regeneration of plants from plant tissues. The particular method of regeneration will depend on the starting plant tissue and the particular plant species to be regenerated. The regeneration, development and cultivation of plants from single plant protoplast transformants or from various transformed explants is well known in the art (Weissbach and Weissbach, Eds.; In Methods for Plant Molecular Biology; Academic Press, Inc.: San Diego, Calif., 1988). This regeneration and growth process typically includes the steps of selection of transformed cells, culturing those individualized cells through the usual stages of embryonic development or through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

Another general application of the EME and TATA box sequences of the disclosure is to construct chimeric polynucleotides that can be used to increase or reduce expression of at least one heterologous nucleic acid fragment in a plant cell. To accomplish this, a chimeric gene designed for gene silencing of a heterologous nucleic acid fragment can be constructed by linking the fragment to the EME and TATA box sequences of the present disclosure. Alternatively, a chimeric gene designed to express antisense RNA for a heterologous nucleic acid fragment can be constructed by linking the fragment in reverse orientation to the EME and TATA box sequences of the present disclosure. Either the co-suppression or antisense chimeric gene can be introduced into plants via transformation. Transformants wherein expression of the heterologous nucleic acid fragment is decreased or eliminated are then selected.

This disclosure also concerns a method of altering (increasing or decreasing) the expression of at least one heterologous nucleic acid fragment in a plant cell which comprises:
(a) transforming a plant cell with the recombinant expression construct described herein;
(b) growing fertile mature plants from the transformed plant cell of step (a);
(c) selecting plants containing a transformed plant cell wherein the expression of the heterologous nucleic acid fragment is increased or decreased.

Transformation and selection can be accomplished using methods well-known to those skilled in the art including, but not limited to, the methods described herein.

In an embodiment, the EME and TATA box sequences are present within about 10 to about 5000 bp from a transcriptional start site of the endogenous polynucleotide. This location range also includes about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 1000, 2000, 3000, 4000 and 5000 nucleotides from the TSS. In another embodiment, the EME and TATA box sequences are present within about 10 to about 5000 bp from a start codon of the endogenous polynucleotide. This location range also includes about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 1000, 2000, 3000, 4000 and 5000 nucleotides from the start codon. In an embodiment, the EME and TATA box sequences further comprise additional copies of the expression modulating element such that about 2× to 10× copies of the EMEs or TATA box sequences are present in the regulatory region of the endogenous polynucleotide or a recombinant polynucleotide. Additional number of copies such as 3×, 4×, 5×, 6×, 7×, 8×, 9× are also suitable based on the need to express a particular polynucleotide higher or lower depending upon e.g., a trait of interest. In another embodiment, the EME and TATA box sequences further comprise additional copies of TATA box sequence such that about 2× to 10× copies of the TATA box sequences are present in the regulatory region of the endogenous polynucleotide or a recombinant polynucleotide. Additional number of copies such as 3×, 4×, 5×, 6×, 7×, 8×, 9× are also suitable based on the need to express a particular polynucleotide higher or lower depending upon e.g., a trait of interest.

In an embodiment, when more than one copy of the EME or TATA box is present, it can be present in one or more of the configurations selected from the group consisting of: head to head, head to tail, tail to head, tail to tail, and a combination thereof. In an embodiment, the additional copies are separated by a spacer sequence, which may include about 1 to 50 nucleotides. In an embodiment, the EME or TATA box is a combination of one or more copies of heterologous expression elements. Suitable length of a spacer that is present between one or more EME or TATA box of the present disclosure include for example, about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100 or more contiguous polynucleotides. The spacer sequences may include intron elements or other non-coding sequences that do not materially alter the function intended to be conveyed by the EME or TATA box.

### EXAMPLES

The present disclosure is further defined in the following Examples, in which parts and percentages are by weight and degrees are Celsius, unless otherwise stated. Sequences of promoters, cDNA, adaptors, and primers listed in this disclosure all are in the 5′ to 3′ orientation unless described otherwise. It should be understood that these Examples, while indicating preferred embodiments of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various usages and conditions. Thus, various modifications of the disclosure in addition to those shown and described herein will be apparent to those skilled in the art from the foregoing description. Such modifications are also intended to fall within the scope of the appended claims.

The disclosure of each reference set forth herein is incorporated herein by reference in its entirety.

### Example 1

### Assay for Quantification of Reporter Gene

Transfection vectors with a reporter gene (e.g., ZsGreen) were constructed with TATA box sequences and/or expression modulating elements (EMEs). Those vectors were tested in maize leaf protoplasts. This protoplast expression assay uses a modified version of this commonly used protocol to facilitate the delivery of known plasmid DNA to cells isolated from maize inbred leaf mesophyll cells. The transfection method utilized in this assay was the polyethelene glycol 40% w/v mediated transfection.

The quantification methodology used in the protoplast expression assay was based around the BioTek Cytation5 inverted microscope imager. Images are taken of the transfected protoplast populations using excitation and emission spectra as determined based on the fluorescent markers chosen for the experiment. When quantification of a known element was required, a dual cassette expression vector was used. The normalization cassette consists of a strong constitutive promoter Seteria UBI along with Seteria UBI intron driving TagRFP; this cassette also acts as a transfection control to monitor transfection efficiency. The experimental cassette contains the DNA sequence being evaluated with ZsGreen as the reporter gene. Post imaging processing was carried out primarily in the BioTek Gen5 software. Using a circularity, size, and presence of TagRFP fluorescence algorithm, positively transfected cells were identified and the relative fluorescence based on pixel intensity was recorded. The fluorescence recorded from the GFP channel was normalized to the RFP in order to quantify on a cell by cell basis. The geometric mean was calculated for each experimental entity and compared to the appropriate control with 95% confidence intervals.

### Example 2

### TATA Box Optimization and Multimer Effects

Native TATA box sequence and optimized TATA box sequences were tested to determine effects of TATA box sequence optimizations. Further, several configurations of TATA box sequences were tested to determine effects of TATA sequence variations and multimers of the TATA box sequences.

In PROMOTER 1, two potential TATA box sequences were identified. The TATA box at −83 of the ATG (start codon) was already optimal (i.e. TATA), while the second candidate at −130 of the ATG was sub-optimal (TACA). Optimal TATA sequences and 3× multimers of the optimal sequence were tested at each location. Data is shown in Table 1 below.

TABLE 1
PROMOTER 1 (PRO 1) TATA optimization

| | | | | | | | | | | Fold |
|---|---|---|---|---|---|---|---|---|---|---|
| | | | | | | | | | | difference |
| | | | | TATA | TATA | | | | | over |
| | | | | sequence | sequence | Geo- | Lower | Upper | | control |
| | Plasmid | | | at -130 | at -83 | metric | Error | Error | Sample | (calculated |
| Rep | ID | PRO | TATA description | of ATG | of ATG | Mean | Bar | bar | Size | as %) |
| 1 | EE2360 | PRO 1 | Native (-130), | TACAA | TATAA | 0.3146 | 0.0215 | 0.0231 | 614 | Control |
| | | | Native (-83) | | | | | | | |
| 1 | EE2369 | PRO 1 | Native (-130), | TACAA | TATATATA | 0.1864 | 0.0049 | 0.0050 | 3004 | 0.59 |
| | | | 3X TATA (-83) | | TATAA | | | | | |
| 1 | EE2370 | PRO 1 | TATA(Optimized)(-130), | TATAA | TATAA | 0.1802 | 0.0042 | 0.0043 | 3584 | 0.57 |
| | | | Native (-83) | | | | | | | |
| 1 | EE2371 | PRO 1 | 3X TATA(Optimized)(-130), | TATATATA | TATAA | 0.2259 | 0.0077 | 0.0080 | 1630 | 0.72 |
| | | | Native (-83) | TATAA | | | | | | |
| 2 | EE2360 | PRO 1 | Native (-130), Native (-83) | TACAA | TATAA | 0.2545 | 0.0131 | 0.0138 | 1112 | Control |
| 2 | EE2369 | PRO 1 | Native (-130), 3X TATA (-83) | TACAA | TATATATAT | 0.1347 | 0.0035 | 0.0036 | 3103 | 0.53 |
| | | | | | ATAA | | | | | |
| 2 | EE2370 | PRO 1 | TATA(Optimized)(-130), | TATAA | TATAA | 0.1330 | 0.0032 | 0.0033 | 3665 | 0.52 |
| | | | Native (-83) | | | | | | | |
| 2 | EE2371 | PRO 1 | 3X TATA(Optimized)(-130), | TATATATA | TATAA | 0.1390 | 0.0040 | 0.0041 | 2353 | 0.55 |
| | | | Native (-83) | TATAA | | | | | | |

1×, 2×, and 3× multimers of TATA box sequences were tested to determine gene expression modulation driven by PROMOTER 2.

Data is shown in Table 2 below.

TABLE 2
PROMOTER 2 (PRO 2) TATA optimization

| | | | | | | | | | Fold |
|---|---|---|---|---|---|---|---|---|---|
| | | | | | | | | | difference |
| | | | | | | | | | over |
| | | | | | Geo- | Lower | Upper | | control |
| | Plasmid | | TATA | TATA | metric | Error | Error | Sample | (calculated |
| Rep | ID | PRO | description | Sequence | Mean | Bar | bar | Size | as %) |
| 1 | EE2386 | PRO 2 | Native | TATA | 0.1205 | 0.0029 | 0.0030 | 3699 | Control |
| 1 | EE2387 | PRO 2 | 2X TATA | TATATATA | 0.0975 | 0.0028 | 0.0029 | 2284 | 0.81 |
| 1 | EE2388 | PRO 2 | 3X TATA | TATATATATATA | 0.1122 | 0.0031 | 0.0032 | 2606 | 0.93 |
| 2 | EE2386 | PRO 2 | Native | TATA | 0.1215 | 0.0025 | 0.0026 | 4526 | Control |
| 2 | EE2387 | PRO 2 | 2X TATA | TATATATA | 0.1182 | 0.0034 | 0.0035 | 2331 | 0.97 |
| 2 | EE2388 | PRO 2 | 3X TATA | TATATATATATA | 0.1237 | 0.0032 | 0.0033 | 2797 | 1.02 |

Several configurations of optimized TATA box sequences were tested to determine gene expression modulation driven by PROMOTER 3. PROMOTER 3 lacks a consensus TATA box sequence. Two sequences were identified as potential TATA sequences. One at −95 of the ATG and a second at −81 of the ATG. In plasmid EE2374, the AATA sequence present at −81 of the ATG in the native sequence was edited to TATA to introduce an optimized TATA box. In plasmid EE2375, that new TATA sequence was further edited to a 2× TATA sequence (“TATATATA”). In plasmid EE2376, a third copy of TATA sequence was introduced creating “TATATATATATA”. In plasmid EE2436, in addition to the TATA at −81, the TATA candidate sequence at −95 of the ATG was modified from TGTA to TATA. Data is shown in Table 3 below.

TABLE 3
PROMOTER 3 (PRO 3) TATA optimization

| | | | | | | | | | Fold |
|---|---|---|---|---|---|---|---|---|---|
| | | | | | | | | | difference |
| | | | TATA | | | | | | over |
| | | | sequence | TATA | Geo- | Lower | Upper | | control |
| | Plasmid | TATA | (-95 of | sequence | metric | Error | Error | Sample | (calculated |
| Rep | ID | description | ATG) | (-81 of ATG) | Mean | Bar | bar | Size | as %) |
| 1 | EE2020 | Native | TGTA | AATA | 0.2647 | 0.0060 | 0.0061 | 4040 | Control |
| 1 | EE2374 | 1X TATA | TGTA | TATA | 0.1778 | 0.0043 | 0.0044 | 3013 | 0.67 |
| | | (Optimized) | | | | | | | |
| 1 | EE2375 | 2X TATA | TGTA | TATATATA | 0.6430 | 0.0219 | 0.0226 | 1650 | 2.43 |
| | | (Optimized) | | | | | | | |
| 1 | EE2376 | 3X TATA | TGTA | TATATATATATA | 0.5903 | 0.0203 | 0.0211 | 1718 | 2.23 |
| | | (Optimized) | | | | | | | |
| 1 | EE2436 | 1X TATA | TATA | TATA | 0.1845 | 0.0043 | 0.0044 | 2500 | 0.70 |
| | | (Optimized) | | | | | | | |
| 2 | EE2020 | Native | TGTA | AATA | 0.3301 | 0.0100 | 0.0103 | 2478 | Control |
| 2 | EE2374 | 1X TATA | TGTA | TATA | 0.1803 | 0.0048 | 0.0049 | 2606 | 0.55 |
| | | (Optimized) | | | | | | | |
| 2 | EE2375 | 2X TATA | TGTA | TATATATA | 0.6674 | 0.0227 | 0.0235 | 1621 | 2.02 |
| | | (Optimized) | | | | | | | |
| 2 | EE2376 | 3X TATA | TGTA | TATATATATATA | 0.6012 | 0.0238 | 0.0248 | 1431 | 1.82 |
| | | (Optimized) | | | | | | | |
| 2 | EE2436 | 1X TATA | TATA | TATA | 0.1648 | 0.0037 | 0.0038 | 2598 | 0.50 |
| | | (Optimized) | | | | | | | |

Several configurations of native or optimized TATA box sequences were tested to determine gene expression modulation driven by PROMOTER 4. The native TATA box sequence, TAAA, was edited to TATA in plasmid EE2462. In plasmid EE2463, an additional TATA sequence was added adjacent to the TATA sequence of EE2462. Data is shown in Table 4 below.

TABLE 4
PROMOTER 4 (PRO 4) TATA optimization

| | | | | | | | | | | Fold |
|---|---|---|---|---|---|---|---|---|---|---|
| | | | | | | | | | | difference |
| | | | | | | | | | | over |
| | | | | | | Geo- | Lower | Upper | | control |
| | Plasmid | | TATA | | TATA | metric | Error | Error | Sample | (calculated |
| Rep | ID | PRO | description | TATA location | sequence | Mean | Bar | bar | Size | as %) |
| 1 | EE2262 | PRO 4 | Native | -175 of the ATG | TAAA | 0.1453 | 0.0047 | 0.0048 | 1786 | Control |
| 1 | EE2462 | PRO 4 | 1X TATA | -175 of the ATG | TATA | 0.1804 | 0.0055 | 0.0056 | 1905 | 1.24 |
| | | | (Optimized) | | | | | | | |
| 1 | EE2463 | PRO 4 | 2X TATA | -175 of the ATG | TATATATA | 0.1536 | 0.0048 | 0.0049 | 1818 | 1.06 |
| | | | (Optimized) | | | | | | | |
| 2 | EE2262 | PRO 4 | Native | -175 of the ATG | TAAA | 0.1376 | 0.0046 | 0.0048 | 1463 | Control |
| 2 | EE2462 | PRO 4 | 1X TATA | -175 of the ATG | TATA | 0.1292 | 0.0042 | 0.0043 | 1577 | 0.94 |
| | | | (Optimized) | | | | | | | |
| 2 | EE2463 | PRO 4 | 2X TATA | -175 of the ATG | TATATATA | 0.1683 | 0.0057 | 0.0059 | 1408 | 1.22 |
| | | | (Optimized) | | | | | | | |

### Example 3

### EME+TATA Enhancement Experiments

Several configurations of EME and TATA box sequences were tested to determine gene expression modulation driven by different promoters.

Several configurations of the EME2 with native or optimized TATA box sequences were tested to determine gene expression modulation driven by PROMOTER 5. The native TAAA sequence in PROMOTER 5 was edited to TATA to produce an optimized TATA box sequence. Data is shown in Table 5 below.

In PROMOTER 5, the distance between EME and TATA was 21 or 92 bp.

TABLE 5
PROMOTER 5 (PRO 5) expression modulation with EME2 and TATA

| | | | | | | | | | | | Fold |
|---|---|---|---|---|---|---|---|---|---|---|---|
| | | | | | | | | | | | difference |
| | | | EME | | | | | | | | over |
| | | | distance | | | | Geo- | Lower | Upper | | control |
| Plasmid | | | from | TATA | TATA | | metric | Error | Error | Sample | (calculated |
| ID | PRO | EME | TATA (bp) | description | Location | TATA | Mean | Bar | bar | Size | as %) |
| EE1275 | PRO 5 | | | Native | -164 of ATG | TAAA | 0.3178 | 0.0127 | 0.0132 | 974 | Control |
| EE1534 | PRO 5 | EME2 | -92 | Native | -164 of ATG | TAAA | 3.5541 | 0.2656 | 0.2871 | 561 | 11.18 |
| EE1535 | PRO 5 | EME2 | -21 | Native | -164 of ATG | TAAA | 18.8346 | 0.9718 | 1.0247 | 846 | 59.27 |
| EE1536 | PRO 5 | 2X | -21 & -92 | Native | -164 of ATG | TAAA | 31.5984 | 2.0736 | 2.2193 | 539 | 99.43 |
| | | EME2 | | | | | | | | | |
| EE1785 | PRO 5 | EME2 | -21 | 1X TATA | -164 of ATG | TATA | 28.8533 | 1.1099 | 1.1543 | 1045 | 90.79 |
| | | | | (Optimized) | | | | | | | |

Several configurations of native or optimized TATA box sequences with, or without a 2× EME2 were tested to determine gene expression modulation driven by PROMOTER 3. The native AATA sequence in PROMOTER 3 was edited to TATA to produce optimized TATA box sequence, then further edited to TATATATA, as well as TATATATATATA. Data is shown in Table 6 below.

In PROMOTER 3, the distance between EME and TATA was 20 bp and 43 bp.

TABLE 6
PROMOTER 3 (PRO 3) expression modulation with EME2 and TATA

| | | | | EME | | | | | | | Fold difference |
|---|---|---|---|---|---|---|---|---|---|---|---|
| | | | | Distance | | | | Lower | Upper | | over control |
| | Plasmid | | | from TATA | | TATA | Geometric | Error | Error | Sample | (calculated as |
| Rep | ID | PRO | EME | (bp) | TATA description | Sequence | Mean | Bar | bar | Size | %) |
| 1 | EE2020 | PRO 3 | | | Native | AATA | 0.4634 | 0.0161 | 0.0167 | 1548 | Control |
| 1 | EE2375 | PRO 3 | | | 2X TATA(Optimized) | TATATATA | 0.6023 | 0.0240 | 0.0250 | 1174 | 1.30 |
| 1 | EE2381 | PRO 3 | 2X EME2 | -20 and -43 | Native | AATA | 3.4596 | 0.0643 | 0.0655 | 4822 | 7.47 |
| 1 | EE2382 | PRO 3 | 2X EME2 | -20 and -43 | 1X TATA(Optimized) | TATA | 5.9727 | 0.1248 | 0.1275 | 3821 | 12.89 |
| 1 | EE2383 | PRO 3 | 2X EME2 | -20 and -43 | 2X TATA(Optimized) | TATATATA | 59.6801 | 1.4377 | 1.4732 | 2021 | 128.79 |
| 1 | EE2384 | PRO 3 | 2X EME2 | -20 and -43 | 3X TATA(Optimized) | TATATATA | 52.5509 | 0.8666 | 0.8811 | 4616 | 113.40 |
| | | | | | | TATA | | | | | |
| 2 | EE2020 | PRO 3 | | | Native | AATA | 0.4010 | 0.0118 | 0.0122 | 1955 | Control |
| 2 | EE2375 | PRO 3 | | | 2X TATA(Optimized) | TATATATA | 0.6218 | 0.0283 | 0.0297 | 947 | 1.55 |
| 2 | EE2381 | PRO 3 | 2X EME2 | -20 and -43 | Native | AATA | 3.4334 | 0.0650 | 0.0663 | 4652 | 8.56 |
| 2 | EE2382 | PRO 3 | 2X EME2 | -20 and -43 | 1X TATA(Optimized) | TATA | 5.9143 | 0.1394 | 0.1427 | 2851 | 14.75 |
| 2 | EE2383 | PRO 3 | 2X EME2 | -20 and -43 | 2X TATA(Optimized) | TATATATA | 28.5355 | 1.5640 | 1.6547 | 784 | 71.16 |
| 2 | EE2384 | PRO 3 | 2X EME2 | -20 and -43 | 3X TATA(Optimized) | TATATATA | 45.8896 | 0.9220 | 0.9409 | 3500 | 114.44 |
| | | | | | | TATA | | | | | |

Several configurations of native or optimized TATA box sequences or TATA knockout sequences with several configurations of EME2 were tested to determine gene expression modulation driven by PROMOTER 6 (PRO 6) promoter. Data is shown in Table 7 below.

In PROMOTER 6, the nearest distance from EME to TATA was 21 bp.

TABLE 7
PROMOTER 6 (PRO 6) expression modulation with EME2 and TATA

| | | | | | | | | Lower | Upper | | Fold difference |
|---|---|---|---|---|---|---|---|---|---|---|---|
| | Plasmid | | | EME distance | TATA | | Geometric | Error | Error | Sample | over control |
| Rep | ID | PRO | EME | from TATA (bp) | description | TATA Sequence | Mean | Bar | bar | Size | (calculated as %) |
| 1 | EE1619 | PRO 6 | | | TATA(Native) | TATA | 5.3812 | 0.258 | 0.271 | 1872 | Control |
| 1 | EE2430 | PRO 6 | | | TATA Knockout | AGTC | 2.2299 | 0.1022 | 0.1071 | 443 | 0.41 |
| 1 | EE2807 | PRO 6 | | | 2X TATA | TATATATA | 7.4239 | 0.2129 | 0.2192 | 1817 | 1.38 |
| 1 | EE2431 | PRO 6 | 2X EME2 | -21 | TATA Knockout | AGTC | 5.2791 | 0.1416 | 0.1455 | 2021 | 0.98 |
| 1 | EE2208 | PRO 6 | 2X EME2 | -21 | TATA(Native) | TATA | 32.3922 | 0.6711 | 0.6853 | 2042 | 6.02 |
| 1 | EE2808 | PRO 6 | 2X EME2 | -21 | 2X TATA | TATATATA | 32.82 | 0.628 | 0.6402 | 2008 | 6.10 |
| 1 | EE2809 | PRO 6 | 2X EME2 | -21 | 3X TATA | TATATATATATA | 30.8684 | 0.6514 | 0.6655 | 2111 | 5.74 |
| 2 | EE1619 | PRO 6 | | | TATA(Native) | TATA | 3.7344 | 0.2319 | 0.2472 | 1124 | Control |
| 2 | EE2430 | PRO 6 | | | TATA Knockout | AGTC | 2.3345 | 0.1412 | 0.1503 | 161 | 0.63 |
| 2 | EE2807 | PRO 6 | | | 2X TATA | TATATATA | 5.6805 | 0.1945 | 0.2014 | 1826 | 1.52 |
| 2 | EE2431 | PRO 6 | 2X EME2 | -21 | TATA Knockout | AGTC | 3.934 | 0.111 | 0.1142 | 2130 | 1.05 |
| 2 | EE2208 | PRO 6 | 2X EME2 | -21 | TATA(Native) | TATA | 34.6927 | 0.6681 | 0.6813 | 2083 | 9.29 |
| 2 | EE2808 | PRO 6 | 2X EME2 | -21 | 2X TATA | TATATATA | 34.7385 | 0.7489 | 0.7654 | 1883 | 9.30 |
| 2 | EE2809 | PRO 6 | 2X EME2 | -21 | 3X TATA | TATATATATATA | 30.775 | 0.6526 | 0.6668 | 2233 | 8.24 |

Several configurations of EME2 with native (TAAA) or optimized TATA box sequence were tested to determine gene expression modulation driven by PROMOTER 4. Data is shown in Table 8 below.

In PROMOTER 4, the distance between EME and TATA was 92 bp or 21 bp.

TABLE 8
PROMOTER 4 (PRO 4) expression modulation with EME2 and TATA

| | | | | EME Distance | | | | Lower | Upper | Sample | Fold difference over |
|---|---|---|---|---|---|---|---|---|---|---|---|
| | Plasmid | | | from TATA | | TATA | Geometric | Error | Error | Size | control (calculated |
| Rep | ID | Promoter | EME | (bp) | TATA description | sequence | Mean | Bar | bar | | as %) |
| 1 | EE2262 | PRO 4 | | | Native | TAAA | 0.1315 | 0.0025 | 0.0025 | 4970 | Control |
| 1 | EE2265 | PRO 4 | 1X EME2 | -92 | Native | TAAA | 3.0950 | 0.0654 | 0.0668 | 2443 | 23.54 |
| 1 | EE2268 | PRO 4 | 2X EME2 | -21 & -92 | Native | TAAA | 14.1592 | 0.2460 | 0.2504 | 3274 | 107.67 |
| 1 | EE2272 | PRO 4 | 1X EME2 | -92 | 1X TATA | TATA | 5.9066 | 0.1505 | 0.1544 | 2171 | 44.92 |
| | | | | | (Optimized) | | | | | | |
| 1 | EE2275 | PRO 4 | 2X EME2 | -21 & -92 | 1X TATA | TATA | 29.9923 | 0.4257 | 0.4318 | 4567 | 228.08 |
| | | | | | (Optimized) | | | | | | |
| 2 | EE2262 | PRO 4 | | | Native | TAAA | 0.1515 | 0.0030 | 0.0030 | 4525 | Control |
| 2 | EE2265 | PRO 4 | 1X EME2 | -92 | Native | TAAA | 3.1997 | 0.0608 | 0.0620 | 2899 | 21.12 |
| 2 | EE2268 | PRO 4 | 2X EME2 | -21 & -92 | Native | TAAA | 16.4816 | 0.2858 | 0.2908 | 3282 | 108.79 |
| 2 | EE2272 | PRO 4 | 1X EME2 | -92 | 1X TATA | TATA | 4.6688 | 0.1407 | 0.1451 | 1523 | 30.82 |
| | | | | | (Optimized) | | | | | | |
| 2 | EE2275 | PRO 4 | 2X EME2 | -21 & -92 | 1X TATA | TATA | 31.7218 | 0.4812 | 0.4886 | 3996 | 209.39 |
| | | | | | (Optimized) | | | | | | |

Several configurations of EME2 with several configurations of native or optimized TATA box sequence were tested to determine gene expression modulation driven by PROMOTER 1. The native TACA sequence in PROMOTER 1 was edited to TATA to produce an optimized TATA box sequence.

In PROMOTER 1, the distance between nearest EME and TATA was 20 bp.

Data is shown in Table 9 below.

TABLE 9
PROMOTER 1 (PRO 1) TATA variations with EME2

| | | | | | | | | Lower | Upper | | Fold difference |
|---|---|---|---|---|---|---|---|---|---|---|---|
| | Plasmid | | | EME distance | | | Geometric | Error | Error | Sample | over control |
| Rep | ID | PRO | EME | from TATA | TATA description | TATA Sequence | Mean | Bar | bar | Size | (calculated as %) |
| 1 | EE2360 | PRO 1 | | | Native | TACA | 0.3146 | 0.0215 | 0.0231 | 614 | Control |
| 1 | EE2373 | PRO 1 | 2X EME2 | -20 and -42 | 3X TATA(Optimized) | TATATATATATA | 25.6272 | 0.4779 | 0.4869 | 3319 | 81.46 |
| 1 | EE2370 | PRO 1 | | | 1X TATA(Optimized) | TATA | 0.1802 | 0.0042 | 0.0043 | 3584 | 0.57 |
| 1 | EE2366 | PRO 1 | IX EME2 | -20 | 1X TATA(Optimized) | TATA | 6.3742 | 0.1632 | 0.1675 | 2285 | 20.26 |
| 1 | EE2367 | PRO 1 | 2X EME2 | -20 and -42 | 1X TATA(Optimized) | TATA | 23.0697 | 0.4533 | 0.4623 | 2539 | 73.33 |
| 1 | EE2368 | PRO 1 | 3X EME2 | -20, -42, and -67 | 1X TATA(Optimized) | TATA | 18.0495 | 0.9355 | 0.9866 | 908 | 57.37 |
| 1 | EE2362 | PRO 1 | 2X EME2 | -20 and -42 | Native | TACA | 12.2710 | 0.2878 | 0.2947 | 2049 | 39.01 |
| 1 | EE2371 | PRO 1 | | | 3X TATA(Optimized) | TATATATATATA | 0.2259 | 0.0077 | 0.0080 | 1630 | 0.72 |
| 2 | EE2360 | PRO 1 | | | Native | TACA | 0.2545 | 0.0131 | 0.0138 | 1112 | Control |
| 2 | EE2373 | PRO 1 | 2X EME2 | -20 and -42 | 3X TATA(Optimized) | TATATATATATA | 24.1647 | 0.4346 | 0.4426 | 3797 | 94.95 |
| 2 | EE2370 | PRO 1 | | | 1X TATA(Optimized) | TATA | 0.1330 | 0.0032 | 0.0033 | 3665 | 0.52 |
| 2 | EE2366 | PRO 1 | IX EME2 | -20 | 1X TATA(Optimized) | TATA | 6.7150 | 0.1767 | 0.1815 | 2075 | 26.38 |
| 2 | EE2367 | PRO 1 | 2X EME2 | -20 and -42 | 1X TATA(Optimized) | TATA | 26.4102 | 0.5537 | 0.5655 | 2405 | 103.77 |
| 2 | EE2368 | PRO 1 | 3X EME2 | -20, -42, and -67 | 1X TATA(Optimized) | TATA | 11.7937 | 0.9642 | 1.0500 | 544 | 46.34 |
| 2 | EE2362 | PRO 1 | 2X EME2 | -20 and -42 | Native | TACA | 17.1063 | 0.3678 | 0.3759 | 2496 | 67.22 |
| 2 | EE2371 | PRO 1 | | | 3X TATA(Optimized) | TATATATATATA | 0.1390 | 0.0040 | 0.0041 | 2353 | 0.55 |

### Example 4

### Endogenous Gene Expression Modulation Through Genome Editing

In an embodiment, the regulatory elements set forth or fragments thereof or variants thereof, and compositions comprising said sequences, can be inserted in operable linkage with an endogenous gene by genome editing using a double-stranded break inducing agent, such as a guided Cas9 endonuclease. Based on the availability of the genetic loci sequence information guide RNAs are designed to target a particular endogenous gene. For example, maize genes involved in improving agronomic characteristics of a maize plant are suitable targets.

In an embodiment, specific point mutations, insertions or deletions of the regulatory elements set forth in the present disclosure, or fragments thereof, or variants thereof, are made in an endogenous polynucleotide in a site-specific manner to introduce or remove regulatory elements described herein. For example, a few mutations can recreate a regulatory element in an endogenous gene that is involved in yield increase or drought tolerance by genome editing using a double-stranded break inducing agent, such as a guided Cas9 endonuclease. Based on the availability of the genetic loci sequence information guide RNAs are designed to target a particular endogenous gene.

Guided Cas9 endonucleases are derived from CRISPR loci (Clustered Regularly Interspaced Short Palindromic Repeats) (also known as SPIDRs—SPacer Interspersed Direct Repeats) which are a family of recently described DNA loci. CRISPR loci are characterized by short and highly conserved DNA repeats (typically 24 to 40 bp, repeated from 1 to 140 times—also referred to as CRISPR-repeats) which are partially palindromic.

Cas endonuclease relates to a Cas protein encoded by a Cas gene, wherein the Cas protein is capable of introducing a double strand break into a DNA target sequence. The Cas endonuclease is guided by a guide polynucleotide to recognize and optionally introduce a double strand break at a specific target site into the genome of a cell (U.S. Application Publication No. 2015/0082478). The guide polynucleotide/Cas endonuclease system includes a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide RNA if a correct protospacer-adjacent motif (PAM) is approximately oriented at the 3′ end of the target sequence.

In one embodiment, the methods comprise modifying the expression of an endogenous gene in a cell by introducing the regulatory elements herein in operable linkage with an endogenous gene. The regulatory elements can be introduced in operable linkage to an endogenous gene using any genome editing technique, including, but not limited to use of a double-stranded break inducing agent, such as guided Cas9/CRISPR system, Zinc finger nucleases, TALENs. See Ma et al (2014), Scientific Reports, 4: 4489; Daimon et al (2013), Development, Growth, and Differentiation, 56(1): 14-25; and Eggleston et al (2001) BMC Genetics, 2:11.

### Example 5

### Gene Expression Modulation by EME and TATA Box Sequences in Plants for Trait Development

A transgenic expression construct is created, containing EME and/or TATA box sequence as part of a regulatory sequence driving expression of a gene of interest. In an embodiment, a heterologous promoter is engineered to have one or more copies of EME in addition to one or more copies of plant optimized TATA sequence. Plants are transformed and then positive transformants are selected. Transgenic seedlings are processed to evaluate expression of the gene and expression of the gene is quantified.

### Example 6

### TATA Variations in the Maize PROMOTER 7 Modulates Expression

The PROMOTER 7 native TATA sequence is “TTTAAATT”. The canonical TATA sequence is “TATAA.” The table below shows that expression can be increased by about 2× by optimizing the sequence to better resemble the canonical TATA sequence. Also shown below is that expression can be reduced by changing the TATA sequence to differ from the canonical TATA sequence, a process also known as de-optimization (deOPT). Through variations in the TATA sequence alone, expression variation in the range of ˜4.5× can be created in maize protoplasts.

Data is shown in Table 10 below.

TABLE 10
TATA variations in maize PROMOTER 7 (PRO 7) (no EME present) in Maize protoplast

| | | | | | | | | | Fold |
|---|---|---|---|---|---|---|---|---|---|
| | | | | | | | | | difference |
| | | | | | | Lower | Upper | | over control |
| | Plasmid | | TATA | TATA | Geometric | Error | Error | Sample | (calculated |
| Rep | ID | PRO | Description | sequence | Mean | Bar | bar | Size | as %) |
| 1 | EE3646 | PRO 7 | Native | TTTAAATT | 8.0013 | 0.2397 | 0.247 | 2319 | Control |
| 1 | EE3647 | PRO 7 | 4 bp deOPT | TTGCGCTT | 3.7464 | 0.0948 | 0.0973 | 3405 | 0.47 |
| 1 | EE3648 | PRO 7 | 8 bp deOPT | GCGCGCGC | 3.5979 | 0.0879 | 0.0902 | 3340 | 0.45 |
| 1 | EE3649 | PRO 7 | 4 bp deOPT | GCGCAATT | 3.2675 | 0.0979 | 0.1009 | 2781 | 0.41 |
| 1 | EE3650 | PRO 7 | 4 bp deOPT | TTTAGCGC | 3.6573 | 0.0853 | 0.0874 | 3262 | 0.46 |
| 1 | EE3651 | PRO 7 | 2X TATA | TATATATA | 14.1688 | 0.2884 | 0.2944 | 3470 | 1.77 |
| | | | (Optimized) | | | | | | |
| 1 | EE3652 | PRO 7 | OPT | TTTATATT | 11.2238 | 0.2229 | 0.2274 | 3216 | 1.40 |
| 1 | EE3653 | PRO 7 | OPT | TATAAATT | 14.847 | 0.2811 | 0.2865 | 4007 | 1.86 |
| 1 | EE3654 | PRO 7 | OPT | TTTATATA | 11.7213 | 0.2385 | 0.2435 | 3691 | 1.46 |
| 1 | EE3655 | PRO 7 | 1X TATA | TATATAA | 12.4168 | 0.247 | 0.252 | 3770 | 1.55 |
| | | | (Optimized) | | | | | | |
| 1 | EE3656 | PRO 7 | 1 bp deOPT | TGTATAA | 4.3193 | 0.0993 | 0.1017 | 3706 | 0.54 |
| 1 | EE3657 | PRO 7 | 1 bp deOPT | TAGATAA | 5.9434 | 0.1331 | 0.1361 | 3265 | 0.74 |
| 1 | EE3658 | PRO 7 | 1 bp deOPT | TATGTAA | 4.7396 | 0.1094 | 0.112 | 3275 | 0.59 |
| 1 | EE3659 | PRO 7 | 2 bp deOPT | TAGCTAA | 4.0942 | 0.1024 | 0.105 | 3190 | 0.51 |
| 1 | EE3660 | PRO 7 | 2 bp deOPT | TGCATAA | 4.3777 | 0.098 | 0.1002 | 3701 | 0.55 |
| 1 | EE3661 | PRO 7 | 4 bp deOPT | GCGCTAA | 4.6405 | 0.0983 | 0.1004 | 4069 | 0.58 |
| 1 | EE3662 | PRO 7 | 7 bp deOPT | GCGCGCG | 3.7448 | 0.0898 | 0.092 | 2519 | 0.47 |
| 1 | EE3663 | PRO 7 | 2 bp deOPT | TGTATGA | 4.3474 | 0.0955 | 0.0976 | 3499 | 0.54 |
| 2 | EE3646 | PRO 7 | Native | TTTAAATT | 7.016 | 0.1923 | 0.1977 | 3309 | Control |
| 2 | EE3647 | PRO 7 | 4 bp deOPT | TTGCGCTT | 3.9946 | 0.0958 | 0.0981 | 4068 | 0.57 |
| 2 | EE3648 | PRO 7 | 8 bp deOPT | GCGCGCGC | 3.8447 | 0.0913 | 0.0935 | 3942 | 0.55 |
| 2 | EE3649 | PRO 7 | 4 bp deOPT | GCGCAATT | 3.2512 | 0.0937 | 0.0965 | 3379 | 0.46 |
| 2 | EE3650 | PRO 7 | 4 bp deOPT | TTTAGCGC | 4.1022 | 0.0868 | 0.0887 | 4224 | 0.58 |
| 2 | EE3651 | PRO 7 | 2X TATA | TATATATA | 14.1478 | 0.2759 | 0.2814 | 4293 | 2.02 |
| | | | (Optimized) | | | | | | |
| 2 | EE3652 | PRO 7 | OPT | TTTATATT | 11.4397 | 0.2325 | 0.2373 | 3540 | 1.63 |
| 2 | EE3653 | PRO 7 | OPT | TATAAATT | 14.6168 | 0.306 | 0.3126 | 4016 | 2.08 |
| 2 | EE3654 | PRO 7 | OPT | TTTATATA | 12.4187 | 0.2544 | 0.2597 | 3854 | 1.77 |
| 2 | EE3655 | PRO 7 | 1X TATA | TATATAA | 13.1087 | 0.2677 | 0.2732 | 3724 | 1.87 |
| | | | (Optimized) | | | | | | |
| 2 | EE3656 | PRO 7 | 1 bp deOPT | TGTATAA | 4.7668 | 0.1122 | 0.1149 | 3693 | 0.68 |
| 2 | EE3657 | PRO 7 | 1 bp deOPT | TAGATAA | 6.3461 | 0.1427 | 0.146 | 3740 | 0.90 |
| 2 | EE3658 | PRO 7 | 1 bp deOPT | TATGTAA | 5.0352 | 0.1218 | 0.1249 | 3666 | 0.72 |
| 2 | EE3659 | PRO 7 | 2 bp deOPT | TAGCTAA | 4.5808 | 0.1013 | 0.1036 | 4194 | 0.65 |
| 2 | EE3660 | PRO 7 | 2 bp deOPT | TGCATAA | 4.5144 | 0.1002 | 0.1025 | 4232 | 0.64 |
| 2 | EE3661 | PRO 7 | 4 bp deOPT | GCGCTAA | 5.2207 | 0.1114 | 0.1138 | 4290 | 0.74 |
| 2 | EE3662 | PRO 7 | 7 bp deOPT | GCGCGCG | 4.6863 | 0.1058 | 0.1083 | 3186 | 0.67 |
| 2 | EE3663 | PRO 7 | 2 bp deOPT | TGTATGA | 4.6897 | 0.1031 | 0.1054 | 3918 | 0.67 |

### Example 7

### TATA Variations in the Maize PROMOTER 8 (No EME Present) in Maize Protoplast

The PROMOTER 8 (PRO 8) native TATA sequence is “TAATAAATA”. The canonical TATA sequence is “TATAA.” The table below shows that expression can be increased by ˜34% with sequences that better resemble the canonical TATA sequence. In contrast, when in a TATA “knockout” wherein the native TATA sequence is replaced with “GCGCGCGC”, expression was reduced by ˜75%.

Alteration of the first nucleotide of the “TAATAAATA” sequence produced the least effect in expression levels, while alteration of the third nucleotide produced the greatest effect in expression levels. G and C substitutions in the TATA sequence appeared to have a greater negative effect than A and T substitutions.
Data is shown in Table 11 below.

TABLE 11
TATA variations in maize PROMOTER 8 (PRO 8) (no EME present) in Maize protoplast

| | | | | | | | | | Fold |
|---|---|---|---|---|---|---|---|---|---|
| | | | | | | | | | difference |
| | | | | | | Lower | Upper | | over control |
| | Plasmid | | TATA | TATA | Geometric | Error | Error | Sample | (calculated |
| Rep | ID | PRO | Description | sequence | Mean | Bar | bar | Size | as %) |
| 1 | EE2954 | PRO 8 | Native | TAATAAATA | 281.1018 | 9.2752 | 9.5917 | 1192 | Control |
| 1 | EE2955 | PRO 8 | 2X TATA | TATATATAA | 353.6419 | 5.325 | 5.4064 | 4059 | 1.26 |
| 1 | EE3026 | PRO 8 | 1X TATA | TATAAA | 265.4287 | 4.3802 | 4.4537 | 3800 | 0.94 |
| 1 | EE3027 | PRO 8 | C4 | TATCAA | 91.2918 | 1.4823 | 1.5067 | 4409 | 0.32 |
| 1 | EE3028 | PRO 8 | C3 | TACAAA | 138.6684 | 2.3581 | 2.3989 | 4474 | 0.49 |
| 1 | EE3029 | PRO 8 | C2 | TCTAAA | 111.9933 | 2.0575 | 2.096 | 4131 | 0.40 |
| 1 | EE3030 | PRO 8 | C1 | CATAAA | 212.633 | 4.4012 | 4.4942 | 2778 | 0.76 |
| 1 | EE3031 | PRO 8 | G4 | TATGAA | 89.8409 | 1.409 | 1.4315 | 4406 | 0.32 |
| 1 | EE3032 | PRO 8 | G3 | TAGAAA | 108.3815 | 2.1043 | 2.1459 | 3204 | 0.39 |
| 1 | EE3033 | PRO 8 | G2 | TGTAAA | 113.033 | 2.0955 | 2.1351 | 3122 | 0.40 |
| 1 | EE3034 | PRO 8 | G1 | GATAAA | 171.5078 | 3.1793 | 3.2394 | 3116 | 0.61 |
| 1 | EE3035 | PRO 8 | T4 | TATTAA | 223.3616 | 3.2817 | 3.3307 | 4936 | 0.79 |
| 1 | EE3036 | PRO 8 | A3 | TAAAAA | 98.0846 | 1.7613 | 1.7935 | 3954 | 0.35 |
| 1 | EE3037 | PRO 8 | T2 | TTTAAA | 250.4834 | 4.929 | 5.028 | 3061 | 0.89 |
| 1 | EE3038 | PRO 8 | A1 | AATAAA | 185.2186 | 2.3754 | 2.4062 | 5731 | 0.66 |
| 1 | EE2956 | PRO 8 | TATA Knockout | GCGCGCGCA | 64.547 | 1.2155 | 1.2388 | 3555 | 0.23 |
| 2 | EE2954 | PRO 8 | Native | TAATAAATA | 234.217 | 9.9961 | 10.4418 | 892 | Control |
| 2 | EE2955 | PRO 8 | 2X TATA | TATATATAA | 339.6808 | 5.1806 | 5.2608 | 4439 | 1.45 |
| 2 | EE3026 | PRO 8 | 1X TATA | TATAAA | 261.979 | 4.0518 | 4.1155 | 4121 | 1.12 |
| 2 | EE3027 | PRO 8 | C4 | TATCAA | 98.4235 | 1.5688 | 1.5942 | 4168 | 0.42 |
| 2 | EE3028 | PRO 8 | C3 | TACAAA | 158.2813 | 3.5087 | 3.5883 | 2586 | 0.68 |
| 2 | EE3029 | PRO 8 | C2 | TCTAAA | 113.4226 | 1.9337 | 1.9673 | 4068 | 0.48 |
| 2 | EE3030 | PRO 8 | C1 | CATAAA | 208.4598 | 4.282 | 4.3719 | 2948 | 0.89 |
| 2 | EE3031 | PRO 8 | G4 | TATGAA | 110.9567 | 1.8492 | 1.8806 | 3881 | 0.47 |
| 2 | EE3032 | PRO 8 | G3 | TAGAAA | 117.1731 | 2.1552 | 2.1956 | 3295 | 0.50 |
| 2 | EE3033 | PRO 8 | G2 | TGTAAA | 104.108 | 1.8595 | 1.8933 | 3401 | 0.44 |
| 2 | EE3034 | PRO 8 | G1 | GATAAA | 164.3671 | 3.1427 | 3.2039 | 2982 | 0.70 |
| 2 | EE3035 | PRO 8 | T4 | TATTAA | 216.804 | 3.4599 | 3.516 | 4326 | 0.93 |
| 2 | EE3036 | PRO 8 | A3 | TAAAAA | 104.3928 | 2.3873 | 2.4431 | 2563 | 0.45 |
| 2 | EE3037 | PRO 8 | T2 | TTTAAA | 232.4322 | 3.8033 | 3.8666 | 4236 | 0.99 |
| 2 | EE3038 | PRO 8 | A1 | AATAAA | 202.439 | 3.8641 | 3.9393 | 3284 | 0.86 |
| 2 | EE2956 | PRO 8 | TATA Knockout | GCGCGCGCA | 68.8722 | 1.3959 | 1.4248 | 3047 | 0.29 |

### Example 8

### TATA Variations in Maize PROMOTER 6 (No EME Present) in Maize Protoplast

The PROMOTER 6 (PRO 6) native TATA is “TATAAA”. About 12 bp upstream is another AT rich region that can interfere with the TATA-box based transcription. For the bulk of the test constructs, the upstream sequence was changed to GCGCGCGCT to see the impact of site-directed changes. This modification increased expression ˜34%. Knocking out the TATA with GCGC reduced expression ˜75%. Alteration of the first nucleotide of the “TATAAA” sequence produced ˜30% to ˜40% reduction in expression. Alteration of the second, third, or fourth nucleotide of the “TATAAA” sequence produced as high as ˜65% reduction in expression. As with PROMOTER 8, G and C substitutions in the TATA sequence have a greater negative effect than A and T substitutions. Data is shown in Table 12 below.

TABLE 12
TATA variations in maize PROMOTER 6 (no EME present) in Maize protoplast

| | | | | | | | | | | | Fold |
|---|---|---|---|---|---|---|---|---|---|---|---|
| | | | | | | | | | | | difference |
| | | | Upstream | Upstream | Downstream | Downstream | | Eower | Upper | | over control |
| | Plasmid | | TATA | TATA | TATA | TATA | Geometric | Error | Error | Sample | (calculated |
| Rep | ID | PRO | description | Sequence | description | Sequence | Mean | Bar | bar | Size | as %) |
| 1 | EE2964 | PRO 6 | TATA Knockout | GCGCGCGCT | Native TATA | TATAAA | 100.2609 | 2.2055 | 2.2551 | 3584 | Control |
| 1 | EE3039 | PRO 6 | TATA Knockout | GCGCGCGCT | C4 | TATCAA | 32.7452 | 0.5916 | 0.6025 | 4143 | 0.33 |
| 1 | EE3040 | PRO 6 | TATA Knockout | GCGCGCGCT | C3 | TACAAA | 64.2871 | 1.3909 | 1.4217 | 3833 | 0.64 |
| 1 | EE3041 | PRO 6 | TATA Knockout | GCGCGCGCT | C2 | TCTAAA | 45.9337 | 0.9054 | 0.9236 | 4240 | 0.46 |
| 1 | EE3042 | PRO 6 | TATA Knockout | GCGCGCGCT | C1 | CATAAA | 74.2207 | 1.3659 | 1.3915 | 4081 | 0.74 |
| 1 | EE3043 | PRO 6 | TATA Knockout | GCGCGCGCT | G4 | TATGAA | 34.1113 | 0.6949 | 0.7094 | 4060 | 0.34 |
| 1 | EE3044 | PRO 6 | TATA Knockout | GCGCGCGCT | G3 | TAGAAA | 36.2111 | 0.7452 | 0.7609 | 4306 | 0.36 |
| 1 | EE3045 | PRO 6 | TATA Knockout | GCGCGCGCT | G2 | TGTAAA | 30.8553 | 0.6734 | 0.6884 | 4173 | 0.31 |
| 1 | EE3046 | PRO 6 | TATA Knockout | GCGCGCGCT | G1 | GATAAA | 55.737 | 1.2378 | 1.266 | 3970 | 0.56 |
| 1 | EE3047 | PRO 6 | TATA Knockout | GCGCGCGCT | T4 | TATTAA | 66.6358 | 1.4178 | 1.4486 | 3701 | 0.66 |
| 1 | EE3048 | PRO 6 | TATA Knockout | GCGCGCGCT | A3 | TAAAAA | 45.5681 | 0.9415 | 0.9613 | 4203 | 0.45 |
| 1 | EE3049 | PRO 6 | TATA Knockout | GCGCGCGCT | T2 | TTTAAA | 59.0412 | 1.3188 | 1.349 | 4176 | 0.59 |
| 1 | EE3050 | PRO 6 | TATA Knockout | GCGCGCGCT | A1 | AATAAA | 67.7028 | 1.4473 | 1.4789 | 3456 | 0.68 |
| 1 | EE2965 | PRO 6 | TATA Knockout | GCGCGCGCT | TATA Knockout | GCGCAA | 25.227 | 0.5537 | 0.5661 | 3511 | 0.25 |
| 1 | EE2430 | PRO 6 | Native TATA | TAATTATAT | TATA Knockout | AGTCAA | 28.6265 | 1.8354 | 1.9612 | 435 | 0.29 |
| 1 | EE2807 | PRO 6 | Native TATA | TAATTATAT | 2X TATA | TATATATAAA | 74.7314 | 1.3883 | 1.4145 | 3919 | 0.75 |
| | | | | | (Optimized) | | | | | | |
| 2 | EE2964 | PRO 6 | TATA Knockout | GCGCGCGCT | Native TATA | TATAAA | 102.9101 | 2.0766 | 2.1194 | 3816 | Control |
| 2 | EE3039 | PRO 6 | TATA Knockout | GCGCGCGCT | C4 | TATCAA | 33.9634 | 0.7198 | 0.7353 | 3160 | 0.33 |
| 2 | EE3040 | PRO 6 | TATA Knockout | GCGCGCGCT | C3 | TACAAA | 74.1016 | 1.6967 | 1.7365 | 3350 | 0.72 |
| 2 | EE3041 | PRO 6 | TATA Knockout | GCGCGCGCT | C2 | TCTAAA | 49.6993 | 1.0043 | 1.025 | 3783 | 0.48 |
| 2 | EE3042 | PRO 6 | TATA Knockout | GCGCGCGCT | C1 | CATAAA | 73.1161 | 1.4803 | 1.5109 | 3678 | 0.71 |
| 2 | EE3043 | PRO 6 | TATA Knockout | GCGCGCGCT | G4 | TATGAA | 44.382 | 1.0359 | 1.0607 | 3014 | 0.43 |
| 2 | EE3044 | PRO 6 | TATA Knockout | GCGCGCGCT | G3 | TAGAAA | 43.0103 | 0.9661 | 0.9883 | 3623 | 0.42 |
| 2 | EE3045 | PRO 6 | TATA Knockout | GCGCGCGCT | G2 | TGTAAA | 40.3771 | 0.8439 | 0.8619 | 3947 | 0.39 |
| 2 | EE3046 | PRO 6 | TATA Knockout | GCGCGCGCT | G1 | GATAAA | 62.2999 | 1.2024 | 1.2261 | 4062 | 0.61 |
| 2 | EE3047 | PRO 6 | TATA Knockout | GCGCGCGCT | T4 | TATTAA | 67.7212 | 1.4012 | 1.4308 | 3911 | 0.66 |
| 2 | EE3048 | PRO 6 | TATA Knockout | GCGCGCGCT | A3 | TAAAAA | 57.91 | 1.0882 | 1.109 | 3772 | 0.56 |
| 2 | EE3049 | PRO 6 | TATA Knockout | GCGCGCGCT | T2 | TTTAAA | 71.6613 | 1.4602 | 1.4906 | 4065 | 0.70 |
| 2 | EE3050 | PRO 6 | TATA Knockout | GCGCGCGCT | A1 | AATAAA | 71.1045 | 1.3155 | 1.3403 | 4079 | 0.69 |
| 2 | EE2965 | PRO 6 | TATA Knockout | GCGCGCGCT | TATA Knockout | GCGCAA | 28.0555 | 0.5315 | 0.5418 | 4109 | 0.27 |
| 2 | EE2430 | PRO 6 | Native TATA | TAATTATAT | TATA Knockout | AGTCAA | 34.2994 | 3.184 | 3.5099 | 270 | 0.33 |
| 2 | EE2807 | PRO 6 | Native TATA | TAATTATAT | 2X TATA | TATATATAAA | 76.044 | 1.2358 | 1.2563 | 4575 | 0.74 |
| | | | | | (Optimized) | | | | | | |

### Example 9

### PROMOTER 3 EME+TATA (TO Transgenic QRT-PCR Data) Maize TO Transgenic Data, V3 Leaf QRT-PCR

Constructs containing Promoter 3 with EME2 and TATA variations were constructed and transformed into maize for transgenic analysis. QRT-PCR was carried out on leaf samples from TO maize V3 leaf, and also in T1 transgenic Maize V3 leaf. This data is presented in Table 13. A similar set of constructs was tested transiently in protoplasts (Table 6).

In PROMOTER 3, the distance between EME and TATA was 20 bp.

TABLE 13
PROMOTER 3 EME + TATA in transgenic Maize

| | | | | | | | | | | Fold |
|---|---|---|---|---|---|---|---|---|---|---|
| | | | | | | | | | | difference |
| | | | | | | | | | | over control |
| Plasmid | | | | TATA | | QRT-PCR | QRT-PCR | | Sample | (calculated as |
| ID | PRO | EME | TATA Description | sequence | Tissue | Median | Average | Std Dev | size | %) |
| PHP95240 | PRO 3 | | 2X TATA (Optimized) | TATATATA | T0 (Leaf V3) | 0.0083585 | 0.0111743 | 0.00729182 | 3 | 0.69 |
| PHP95374 | PRO 3 | | Native | AATA | T0 (Leaf V3) | 0.00868185 | 0.0162377 | 0.0206169 | 8 | Control |
| PHP95241 | PRO 3 | 2X EME2 | 1X TATA (Optimized) | TATA | T0 (Leaf V3) | 0.00606003 | 0.00870722 | 0.00676577 | 4 | 0.54 |
| PHP95242 | PRO 3 | 2X EME2 | 2X TATA (Optimized) | TATATATA | T0 (Leaf V3) | 0.182368 | 0.219881 | 0.189224 | 20 | 13.54 |
| PHP95243 | PRO 3 | 2X EME2 | 3X TATA (Optimized) | TATATATATATA | T0 (Leaf V3) | 0.0889745 | 0.113027 | 0.0904441 | 22 | 10.17 |
| PHP95239 | PRO 3 | 2X EME2 | Native | AATA | T0 (Leaf V3) | 0.00896461 | 0.00853422 | 0.00439891 | 10 | 0.53 |
| PHP95240 | PRO 3 | | 2X TATA (Optimized) | TATATATA | T1 (LeafV3) | 0.00027842 | 0.0022166 | 0.0111747 | 54 | 0.73 |
| PHP95374 | PRO 3 | | Native | AATA | T1 (LeafV3) | 0.00053503 | 0.00030244 | 0.00053503 | 42 | Control |
| PHP95241 | PRO 3 | 2X EME2 | 1X TATA (Optimized) | TATA | T1 (LeafV3) | 0.00054156 | 0.00130998 | 0.00268716 | 55 | 0.43 |
| PHP95242 | PRO 3 | 2X EME2 | 2X TATA (Optimized) | TATATATA | T1 (LeafV3) | 0.111393 | 0.104529 | 0.0632227 | 47 | 34.56 |
| PHP95243 | PRO 3 | 2X EME2 | 3X TATA (Optimized) | TATATATATATA | T1 (LeafV3) | 0.0716702 | 0.0686315 | 0.0451555 | 52 | 22.69 |
| PHP95239 | PRO 3 | 2X EME2 | Native | AATA | T1 (LeafV3) | 0.0002533 | 0.00067048 | 0.00112574 | 51 | 0.22 |

### Example 10

### PROMOTER 6 EME+TATA (T0 Transgenic QRT-PCR Data)

The expression results in TO transgenic Maize V6 leaf measured using QRT-PCR shown below is similar to results observed with experiments conducted in Maize protoplasts. Here, data from a “No EME, Native TATA” control is not included because it was not available. Data is shown in Table 14 below.

In PROMOTER 6, the distance from EME to TATA was 21 bp.

TABLE 14
PROMOTER 6 EME + TATA in TO transgenic Maize (V6 leaf QRT-PCR)

| | | | | | | | | | Fold |
|---|---|---|---|---|---|---|---|---|---|
| | | | | | | | | | difference |
| | | | | | | | | | over control |
| Plasmid | | | TATA | TATA | QRT-PCR | QRT-PCR | Std | Sample | (calculated |
| ID | PRO | EME | Description | sequence | Median | Average | Dev | size | as %) |
| N/A | PRO 6 | | Native | TATA | N/A | N/A | N/A | N/A | N/A* |
| PHX11993 | PRO 6 | | TATA | GCGC | 109.346 | 126.636 | 62.806 | 30 | |
| | | | Knockout | | | | | | |
| PHX11991 | PRO 6 | | 2X TATA | TATATATA | 19.155 | 33.7576 | 33.982 | 19 | |
| PHX11992 | PRO 6 | 2X EME2 | Native | TATA | 1728.48 | 1863.96 | 674.56 | 30 | Control |
| PHX11994 | PRO 6 | 2X EME2 | TATA | GCGC | 284.525 | 278.454 | 86.51 | 29 | 0.15 |
| | | | Knockout | | | | | | |
| PHX11990 | PRO 6 | 2X EME2 | 2X TATA | TATATATA | 1264.95 | 1444.92 | 551.08 | 30 | 0.78 |
| PHX11989 | PRO 6 | 2X EME2 | 3X TATA | TATATATATATA | 802.992 | 792.64 | 313.97 | 30 | 0.43 |

*Native TATA (No EME) plants were not available

The above description of various illustrated embodiments of the disclosure is not intended to be exhaustive or to limit the scope to the precise form disclosed. While specific embodiments of and examples are described herein for illustrative purposes, various equivalent modifications are possible within the scope of the disclosure, as those skilled in the relevant art will recognize. The teachings provided herein can be applied to other purposes, other than the examples described above. Numerous modifications and variations are possible in light of the above teachings and, therefore, are within the scope of the appended claims.

These and other changes may be made in light of the above detailed description. In general, in the following claims, the terms used should not be construed to limit the scope to the specific embodiments disclosed in the specification and the claims.

The entire disclosure of each document cited (including patents, patent applications, journal articles, abstracts, manuals, books or other disclosures) in the Background, Detailed Description, and Examples is herein incorporated by reference in their entireties.

Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight; temperature is in degrees centigrade; and pressure is at or near atmospheric.

What is claimed is:

1. A method of modulating expression of an endogenous polynucleotide in a genomic locus of a plant cell, the method comprising altering one or more nucleotides in a regulatory region of the genomic locus comprising the endogenous polynucleotide to create a modified TATA box in the regulatory region, wherein the regulatory region further comprises one or more copies of a heterologous expression modulating element.

2. The method of claim 1, wherein the modified TATA box comprises the sequence “TATA”.

3. The method of claim 1, wherein the modified TATA box comprises the sequence “TATATATA”.

4. The method of claim 1, wherein the modified TATA box is about 175 nucleotides from a start codon.

5. The method of claim 1, wherein the modified TATA box is within 100 nucleotides from a transcription start site (TSS).

6. The method of claim 1, wherein the expression modulating element is within 250 nucleotides from the TATA box.

7. The method of claim 1, wherein the altering of one or more nucleotides results in a TATA box comprising the sequence TATA from a different sequence.

8. A method of modulating expression of a polynucleotide encoding a polypeptide in a plant, the method comprising expressing the polynucleotide by operably linking the polynucleotide with a regulatory element, wherein the regulatory element comprises one of more expression modulating element and a modified TATA box, wherein the expression modulating element is heterologous to the polynucleotide and the expression modulating element is heterologous to a promoter functional in the plant.

9. A method of modulating expression of a polynucleotide encoding a polypeptide in a plant, the method comprising expressing the polynucleotide by operably linking the polynucleotide with a regulatory element, wherein the regulatory element comprises one or more expression modulating element and a modified TATA box, wherein the modified TATA box is heterologous to the polynucleotide and the TATA box is heterologous to a promoter functional in the plant.

10. A recombinant DNA construct comprising a regulatory element and a polynucleotide sequence, wherein the regulatory element comprises one or more expression modulating element and a modified TATA box, wherein the expression modulating element is heterologous to the polynucleotide sequence.

11. A plant cell comprising a regulatory element and a polynucleotide sequence, wherein the regulatory element comprises one or more expression modulating element and modified TATA box, wherein the expression modulating element is heterologous to the polynucleotide sequence.

12. A method of modulating the expression of a polynucleotide sequence of interest in a plant, the method comprising expressing the polynucleotide sequence, wherein expression of the polynucleotide sequence is regulated by a heterologous regulatory element, wherein the regulatory element comprises one or more expression modulating element and modified TATA box, wherein the expression modulating element is heterologous to the polynucleotide sequence.

13. An isolated polynucleotide comprising a regulatory element and a polynucleotide sequence, wherein the regulatory element comprises one or more expression modulating element and modified TATA box, wherein the expression modulating element is heterologous to the polynucleotide sequence, wherein the regulatory element is operably linked to the polynucleotide sequence.

14. A method of generating a population of activation tagged plants comprising one or more copies of a regulatory element, the method comprising transforming a plurality of plants with a recombinant expression cassette comprising the one or more copies of the regulatory element as an activation tag, wherein the regulatory element comprises one or more expression modulating element and modified TATA box; and generating the population of plants that comprise the activation tag.

15. A method of modulating expression of an endogenous polynucleotide in a plant cell, the method comprising providing a deaminase polypeptide operably associated with a site-specific DNA binding polypeptide, whereby the deaminase polypeptide engineers one or more base changes such that at least one copy of a regulatory element is created in a regulatory region of the endogenous polynucleotide, wherein the regulatory element comprises one or more expression modulating element and modified TATA box, thereby modulating expression of the endogenous polynucleotide in the plant cell.

16. The method of claim 1, wherein the TATA sequence is changed to differ from a canonical TATA sequence.

17. A method of modulating expression of an endogenous polynucleotide in a genomic locus of a plant cell, the method comprising altering one or more nucleotides in a regulatory region of the genomic locus comprising the endogenous polynucleotide to create a modified TATA box in the regulatory region, wherein creating the modified TATA box results in an increase in expression.

18. A method of modulating expression of an endogenous polynucleotide in a genomic locus of a plant cell, the method comprising altering one or more nucleotides in a regulatory region of the genomic locus comprising the endogenous polynucleotide to create a modified TATA box in the regulatory region, wherein creating the modified TATA box results in decrease in expression.


SEQUENCE LISTING (3 sequences)

SEQ ID NO: 1 - DNA, 13 bp, Artificial Sequence
  Synthesized
tatatatatataa

SEQ ID NO: 2 - DNA, 12 bp, Artificial Sequence
  Synthesized
tatatatatata

SEQ ID NO: 3 - DNA, 10 bp, Artificial Sequence
  Synthesized
tatatataaa